(19) United States
(12) Patent Application Publication
(10) Pub. No.: US 2010/0076239 A1
(43) Pub. Date: Mar. 25, 2010

(54) ASYMMETRIC CYCLOPROPANATION OF ELECTRON-DEFICIENT OLEFINS WITH DIAZO REAGENTS

(75) Inventor: X. Peter Zhang, Tampa, FL (US)

Correspondence Address: BRYAN CAVE LLP, 211 NORTH BROADWAY, SUITE 3600, ST. LOUIS, MO, 63102-2750, US

(73) Assignee: UNIVERSITY OF SOUTH FLORIDA, Tampa, FL (US)

(21) Appl. No.: 12/561,346

(22) Filed: Sep. 17, 2009

Related U.S. Application Data
(63) Continuation-in-part of application No. 12/205,373, filed on Sep. 5, 2008.
(60) Provisional application No. 61/097,717, filed on Sep. 17, 2008.
(60) Provisional application No. 60/970,691, filed on Sep. 7, 2007.

(51) Int. Cl. C07C 5/27

(52) U.S. Cl. 585/365

(57) ABSTRACT

Cobalt-catalyzed asymmetric cyclopropanation of electron-deficient olefins.

### CROSS-REFERENCE TO RELATED APPLICATIONS

This application claims the benefit of U.S. Provisional Application Ser. No. 61/097,717, filed Sep. 17, 2008, which is incorporated herein by reference in its entirety. This application is also a continuation-in-part of U.S. application Ser. No. 12/205,373, filed Sep. 5, 2008, which, in turn, claims priority from U.S. Provisional Application Ser. No. 60/970,691, filed Sep. 7, 2007, both of which are incorporated herein by reference in their entirety.

### STATEMENT OF GOVERNMENT RIGHTS

This invention was made with Government support under grant number NSF 0711024 awarded by the National Science Foundation and grant number 44286-AC1 awarded by the American Chemical Society. The government has certain rights in the invention.

### FIELD OF THE INVENTION

The present invention generally relates to metal-catalyzed cyclopropanation of olefins. More particularly, the present invention relates to a process for asymmetric cyclopropanation of electron-deficient olefins using a cobalt porphyrin complex.

### BACKGROUND OF THE INVENTION

Metal-catalyzed cyclopropanation of olefins with diazo reagents has attracted great research interest because of its fundamental and practical importance. (Lebel et al., Chem. Rev. 2003, 103, 977; Davies H. M. L., Antoulinakis E., Org. React. 2001, 57, 1; Doyle M. P., Forbes D. C., Chem. Rev. 1998, 98, 911; and Padwa A., Krumpe K. E., Tetrahedron 1992, 48, 5385-5453.) The resulting cyclopropyl units are recurrent motifs in biologically important molecules and serve as versatile precursors in organic synthesis. (Pietruszka J., Chem. Rev. 2003, 103, 1051; Wessjohann et al., Chem. Rev. 2003, 103, 1625; Donaldson W. A., Tetrahedron 2001, 57, 8589; and Salaun J., Chem. Rev. 1989, 89, 1247.) In the past two decades, outstanding asymmetric catalytic processes, notably those based on copper, rhodium and ruthenium have been developed to allow for the synthesis of chiral cyclopropane derivatives from olefins with diazoacetates in high yields and high selectivities. (Fritschi et al., Agnew. Chem., Int. Ed. Engl. 1986, 25, 1005; Evans et al., J. Am. Chem. Soc. 1991, 113, 726; Lo et al., J. Am. Chem. Soc. 1998, 120, 10270; Maxwell et al., Organometallics 1992, 11, 645; Doyle et al., J. Am. Chem. Soc. 1993, 115, 9968; Davies et al., J. Am. Chem. Soc. 1996, 118, 6897; Nishiyama et al., J. Am. Chem. Soc. 1994, 116, 2223; and Che et al., J. Am. Chem. Soc. 2001, 123, 4119.)

While a number of catalytic systems worked exceptionally well with styrene derivatives and some electron-rich olefins, asymmetric cyclopropanation of electron-deficient olefins containing electron-withdrawing groups such as α,β-unsaturated carbonyl compounds and nitriles have proven to be a challenging problem presumably due to the electrophilic nature of the metal-carbene intermediates in the catalytic cycles. This catalytic asymmetric process would be highly desirable as the corresponding electrophilic cyclopropanes containing two or more electron-withdrawing groups have shown to be valuable synthetic intermediates for various applications. (Gnad F., Reiser O., Chem. Rev. 2003, 103, 1603; Cativiela C., Diaz-de-Villegas, M. D., Tetrahedron: Asy. 2000, 11, 645; Wong et al., Chem. Rev. 1989, 89, 165; and Danishefsky, Acc. Chem. Res. 1979, 12, 66.)

Among several previous efforts towards metal-catalyzed cyclopropanation of electron-deficient olefins with diazo reagents (Doyle et al., J. Org. Chem. 1980, 45, 1538; Doyle et al., J. Org. Chem. 1982, 47, 4059; Nakamura et al., J. Am. Chem. Soc. 1978, 100, 3443; Denmark et al., J. Org. Chem. 1997, 62, 3375), the most notable example is the (Salen)Ru-based asymmetric catalytic system recently reported by Nguyen and coworkers (Miller et al., Angew. Chem., Int. Ed. 2002, 41, 2953; and Miller et al., Angew. Chem., Int. Ed. 2005, 44, 3885). (For intramolecular asymmetric cyclopropanation of electron-deficient olefins, see: Lin W., Charette A. B., Adv. Synth. Catal. 2005, 347, 1547; for a Cu-catalyzed asymmetric [4+1] cycloaddition of α,β-unsaturated ketones with diazoacetates, see: Son S., Fu G. C., J. Am. Chem. Soc. 2007, 129, 1046; for an organocatalytic process, see: Papageorgiou et al., Agnew. Chem., Int. Ed. Engl. 2003, 42, 828.)

It was shown that methyl methacrylate could be effectively cyclopropanated with ethyl diazoacetate (EDA) using a 5:1 ratio of olefin:EDA, producing the desired product in high yield and high selectivities (both diastereoselectivity and enantioselectivity). However, only moderate results were obtained with acrylonitrile even when the reactions were run in neat olefin.

### SUMMARY OF THE INVENTION

The present invention provides for a general and efficient catalytic system for asymmetric cyclopropanation of electron-deficient olefins. The cobalt (II) complex of the D₂-symmetric chiral porphyrin can cyclopropanate a wide range of electron-deficient olefins, forming the corresponding electrophilic cyclopropane derivatives in high yields and selectivities.

Among the various aspects of the present invention is a process for the asymmetric cyclopropanation of electron-deficient olefins with a cobalt(II) complex of a D₂-symmetric chiral porphyrin [Co(1)].

The present invention is further directed to a process for asymmetric cyclopropanation of an olefin wherein at least one of the olefinic carbon atoms possesses an electron withdrawing group. The process comprises treating the olefin with a diazo ester in the presence of a chiral porphyrin complex.

Other objects and features will be in part apparent and in part pointed out hereinafter.

### ABBREVIATIONS AND DEFINITIONS

The following definitions and methods are provided to better define the present invention and to guide those of ordinary skill in the art in the practice of the present invention. Unless otherwise noted, terms are to be understood according to conventional usage by those of ordinary skill in the relevant art.

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

The terms “alkoxy” or “alkoxyl” as used herein alone or as part of another group denote any univalent radical, RO⁻ where R is an alkyl group.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl, and the like. The substituted alkyl groups described herein may have, as substituents, any of the substituents identified as substituted hydrocarbyl substituents.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl. The substituted aryl groups described herein may have, as substituents, any of the substituents identified as substituted hydrocarbyl substituents.

The terms “diazo” or “azo” as used herein alone or as part of another group denote an organic compound with two linked nitrogen compounds. These moieties include without limitation diazomethane, ethyl diazoacetate, and t-butyl diazoacetate.

The terms “halogen” or “halo” as used herein alone or as part of another group denote chlorine, bromine, fluorine, and iodine.

The term “heteroatom” as used herein denotes atoms other than carbon and hydrogen.

The terms “hydrocarbon” and “hydrocarbyl” as used herein alone or as part of another group denote organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl, and alkynaryl. Unless otherwise indicated, these moieties preferably comprise 1 to 20 carbon atoms.

The term “porphyrin” as used herein denotes a compound comprising a fundamental skeleton of four pyrrole nuclei united through the α positions by four methane groups to form the following macrocyclic structure:

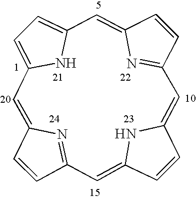

The term “substituted hydrocarbyl” as used herein alone or as part of another group denotes hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substitutents include halogen, heterocyclo, alkoxy, alkenoxy, alkynoxy, aryloxy, hydroxyl, protected hydroxy, keto, acyl, acyloxy, nitro, amino, amido, nitro, cyano, thiol, ketals, acetals, esters and ethers.

### DETAILED DESCRIPTION OF THE PREFERRED EMBODIMENTS

In accordance with the process of the present invention, compounds containing an ethylenic bond, commonly known as olefins, possessing an electron-deficient substituent on at least one of the ethylenic carbons (also sometimes referred to as an olefinic carbon) are cyclopropanated with a diazo reagent in the presence of a cobalt porphyrin complex. Advantageously, the metal porphyrin catalyzed process proceeds relatively efficiently under relatively mild and neutral conditions, in a one-pot fashion, with olefins as limiting reagents and without the need for slow-addition of diazo reagents.

In general, the olefin may be any of a wide range of olefins possessing an electron-deficient substituent on one, or both of the olefinic carbons. One such preferred class of olefins is α,β-unsaturated olefins possessing an electron-withdrawing substituent on the α-olefinic carbon, the β-olefinic carbon, or both. In one embodiment, therefore, the α-olefinic carbon possesses an electron-withdrawing substituent but the β-olefinic carbon does not. In another embodiment, the α-olefinic carbon and the β-olefinic carbon each possess an electron-withdrawing substituent. When the α-olefinic carbon and the β-olefinic carbon each possess an electron-withdrawing substituent, the electron-withdrawing substituents may be in the cis-conformation or the trans-conformation; in certain embodiments, they are preferably in the cis-conformation.

In one embodiment, the olefin corresponds to Formula 1:

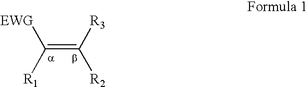

wherein EWG is an electron withdrawing group; R₁ is a substituent of the α-carbon of the ethylenic bond; and R₂ and R₃ are substituents of the β-carbon of the ethylenic bond. Preferably, R₁ is hydrogen, hydrocarbyl, substituted hydrocarbyl, or heterocyclo; and R₂ and R₃ are independently hydrogen, hydrocarbyl, substituted hydrocarbyl, heterocyclo, or an electron withdrawing group. In one embodiment, R₁ is hydrogen. In another embodiment, R₁ is alkyl or substituted alkyl. In one embodiment, R₂ is hydrogen. In another embodiment, R₂ is alkyl or substituted alkyl. In one embodiment, R₃ is hydrogen. In another embodiment, R₃ is alkyl or substituted alkyl. In one embodiment, at least one of R₁, R₂ and R₃ is hydrogen and the other two are alkyl or substituted alkyl. In one embodiment, at least two of R₁, R₂ and R₃ are hydrogen and the other is alkyl or substituted alkyl. In one embodiment, R₂, R₃ and the β-carbon form a carbocyclic or heterocyclic ring. In another embodiment, R₁, R₂, the α-carbon, and the β-carbon form a carbocyclic or heterocyclic ring. In another embodiment, R₁, R₃, the α-carbon, and the β-carbon form a carbocyclic or heterocyclic ring.

When the olefin corresponds to Formula 1 and one of R₂ and R₃ is an electron withdrawing group, the olefin corresponds to Formula 1-trans or 1-cis, respectively:

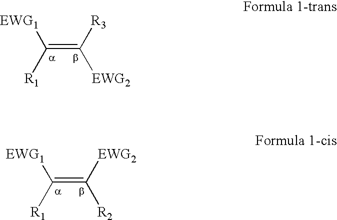

wherein EWG₁ and EWG₂ are electron withdrawing groups and are the same or are different; R₁ is a substituent of the α-carbon of the ethylenic bond; and R₂ and R₃ are substituents of the β-carbon of the ethylenic bond. In this embodiment, R₁, R₂ and R₃ are preferably independently hydrogen, hydrocarbyl, substituted hydrocarbyl or heterocyclo. In one embodiment, R₁, R₃, the α-carbon, and the β-carbon form a carbocyclic or heterocyclic ring. In another embodiment, R₁, R₂, the α-carbon, and the β-carbon form a carbocyclic or heterocyclic ring.

In one preferred embodiment, the olefin corresponds to Formula 1, R₁ is hydrogen, and at least one of R₂ and R₃ is hydrogen. Olefins having this substitution pattern are depicted by Formula 2:

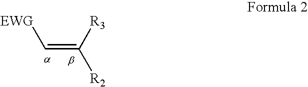

wherein EWG is an electron withdrawing group; and at least one of R₂ and R₃ is hydrogen. In one embodiment, R₂, R₃, and the β-carbon form a carbocylic or heterocyclic ring.

When one of R₂ and R₃ is other than hydrogen, the olefin corresponds to Formula 2-trans or Formula 2-cis:

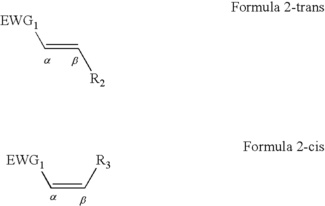

wherein EWG₁ is an electron withdrawing group; R₂ and R₃ are independently hydrogen, hydrocarbyl, substituted hydrocarbyl, heterocyclo, or EWG₂; EWG₂ is an electron withdrawing group; and EWG₁ and EWG₂ are the same or are different.

In another preferred embodiment, the olefin corresponds to Formula 1 and R₁, R₂ and R₃ are hydrogen. Olefins having this substitution pattern are depicted by Formula 3:

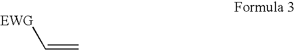

wherein EWG is an electron withdrawing group.

In general, the olefin's electron withdrawing group(s), for example, EWG, EWG₁ or EWG₂ as depicted in Formula 1, Formula 1-trans, Formula 1-cis, Formula 2, Formula 2-trans, Formula 2-cis, or Formula 3, is any substituent that draws electrons away from the ethylenic bond. Exemplary electron withdrawing groups include hydroxy, alkoxy, mercapto, halogens, carbonyls, sulfonyls, nitrile, quaternary amines, nitro, trihalomethyl, imine, amidine, oxime, thioketone, thioester, or thioamide. In one embodiment, the electron withdrawing group(s) is/are hydroxy, alkoxy, mercapto, halogen, carbonyl, sulfonyl, nitrile, quaternary amine, nitro, or trihalomethyl. In another embodiment, the electron withdrawing group(s) is/are halogen, carbonyl, nitrile, quaternary amine, nitro, or trihalomethyl. In another embodiment, the electron withdrawing group(s) is/are halogen, carbonyl, nitrile, nitro, or trihalomethyl. When the electron withdrawing group is alkoxy, it generally corresponds to the formula —OR where R is hydrocarbyl, substituted hydrocarbyl, or heterocyclo. When the electron withdrawing group is mercapto, it generally corresponds to the formula —SR where R is hydrogen, hydrocarbyl, substituted hydrocarbyl or heterocyclo. When the electron withdrawing group is a halogen atom, the electron withdrawing group may be fluoro, chloro, bromo, or iodo; typically, it will be fluoro or chloro. When the electron withdrawing group is a carbonyl, it may be an aldehyde (—C(O)H), ketone (—C(O)R), ester (—C(O)OR), acid (—C(O)OH), acid halide (—C(O)X), amide (—C(O)NRₐR_{b}), or anhydride (—C(O)OC(O)R) where R is hydrocarbyl, substituted hydrocarbyl or heterocyclo, Rₐ, and R_{b} are independently hydrogen, hydrocarbyl, substituted hydrocarbyl or heterocyclo, and X is a halogen atom. When the electron withdrawing group is a sulfonyl, it may be an acid (—SO₃H) or a derivative thereof (—SO₂R) where R is hydrocarbyl, substituted hydrocarbyl or heterocyclo. When the electron withdrawing group is a quaternary amine, it generally corresponds to the formula —N⁺RₐR_{b}R_{c} where Rₐ, R_{b} and R_{c} are independently hydrogen, hydrocarbyl, substituted hydrocarbyl or heterocyclo. When the electron withdrawing group is a trihalomethyl, it is preferably trifluoromethyl or trichloromethyl. In each of the foregoing exemplary electron withdrawing groups containing the variable “X”, in one embodiment, X may be chloro or fluoro, preferably fluoro. In each of the foregoing exemplary electron withdrawing groups containing the variable “R”, R may be alkyl. In each of the foregoing exemplary electron withdrawing groups containing the variable “Rₐ” and “R_{b}”, Rₐ, and R_{b} may independently be hydrogen or alkyl.

In general, α,β-unsaturated carbonyl compounds and α,β-unsaturated nitriles are preferred olefins for cyclopropanation. In one embodiment, therefore, the olefin's electron withdrawing group(s), for example, EWG, EWG₁ or EWG₂ as depicted in Formula 1, Formula 1-trans, Formula 1-cis, Formula 2, Formula 2-trans, Formula 2-cis, or Formula 3, is/are a carbonyl or a nitrile. For other applications, it may nonetheless be preferred that one or both of the ethylenic carbons of the olefin possess a quaternary amine, nitro, or trihalomethyl substituent.

In accordance with one preferred embodiment, the electron withdrawing group(s) is/are a halide, aldehyde, ketone, ester, carboxylic acid, amide, acyl chloride, trifluoromethyl, nitrile, sulfonic acid, ammonia, amine, or a nitro group. In this embodiment, the electron withdrawing group(s) correspond to one of the following chemical structures: —X, —C(O)H, —C(O)R, —C(O)OR, —C(O)OH, —C(O)X, —C(X)₃, —CN, —SO₃H, —N⁺H₃, —N⁺(R)₃, or —N⁺O₂ where R is hydrocarbyl, substituted hydrocarbyl or heterocyclo and X is halogen.

In general, the olefin is cyclopropanated with a carbene. Preferably, the carbene precursor is a diazo reagent (also sometimes referred to herein as a diazo compound) wherein the carbene is generated by the removal of N₂ as nitrogen gas from the solution.

In one preferred embodiment, the carbene precursor is a diazo carbonyl compound. Still more preferably, the carbene precursor is a diazo ester. In some embodiments, the diazo compound is selected from the group consisting of diazo ethylacetate, diazo-t-butylacetate, 2,6-di-tert-butyl-4-methylphenyl diazoacetate, methyl phenyldiazoacetate, ethyl diazoacetacetate, diethyl diazomalonate, and trimethylsilyldiazomethane. In some embodiments, the diazo compound is selected from one of diazo ethylacetate and diazo t-butylacetate. In one preferred embodiment, the diazo compound has the formula N₂CHC(O)OR₁₀ where R₁₀ is hydrocarbyl, substituted hydrocarbyl or heterocyclo. In one such preferred embodiment, the diazo compound has the formula N₂CHC(O)OR₁₀ where R₁₀ is alkyl, aryl or alkaryl, more lower alkyl or aryl. Other exemplary diazo acetates include 2,3,4-trimethyl-3-pentyl diazoacetate, menthyl diazoacetate, 2,5-dimethyl-4-buten-1-yl diazoacetate, 3-(diazoacetyl)amino propionate, and (diazoacetyl)amino acetate.

In one preferred embodiment, the diazo reagent is an α-nitro diazo reagent. More preferably, the diazo reagent is an α-nitro diazo carbonyl compound. Still more preferably, the diazo reagent is an α-nitro diazo ester. In one embodiment, the diazo reagent is an acceptor/acceptor-substituted α-nitro diazo reagent. In one preferred embodiment, the diazo reagent is an α-nitro diazoacetate, corresponding to the following structure:

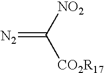

wherein R₁₇ is hydrogen, hydrocarbyl, substituted hydrocarbyl or heterocyclo. In one embodiment, R₁₇ is alkyl, aryl or alkaryl, more lower alkyl or aryl. In one embodiment, R₁₇ is alkyl. In some embodiments, the diazo compound is selected from the group consisting of the α-nitro analogs of diazo ethylacetate, diazo-t-butylacetate, 2,6-di-tert-butyl-4-methylphenyl diazoacetate, methyl phenyldiazoacetate, ethyl diazoacetacetate, diethyl diazomalonate, and trimethylsilyldiazomethane. In some embodiments, the diazo compound is selected from one of the α-nitro analogs of diazo ethylacetate and diazo t-butylacetate. In one preferred embodiment, the diazo compound has the formula N₂C(NO₂)C(O)OR₁₇ where R₁₇ is hydrocarbyl, substituted hydrocarbyl or heterocyclo. In one such preferred embodiment, the diazo compound has the formula N₂C(NO₂)C(O)OR₁₇ where R₁₇ is alkyl, aryl or alkaryl, typically lower alkyl or aryl. Other exemplary diazo acetates include the α-nitro analogs of 2,3,4-trimethyl-3-pentyl diazoacetate, menthyl diazoacetate, 2,5-dimethyl-4-buten-1-yl diazoacetate, 3-(diazoacetyl)amino propionate, and (diazoacetyl)amino acetate.

In another preferred embodiment, the carbene precursor is a diazosulfone. In general, the diazosulfone is selected from the group consisting of aromatic diazosulfones and non-aromatic diazosulfones. In one preferred embodiment, the diazosulfone corresponds to the following structure:

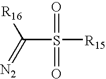

wherein R₁₅ and R₁₆ are independently hydrogen, hydrocarbyl or substituted hydrocarbyl. In one embodiment, R₁₆ is hydrogen and R₁₅ is hydrocarbyl or substituted hydrocarbyl; for example, in this embodiment, R₁₆ is hydrogen and R₁₅ may be alkyl, alkenyl, alkynyl, phenyl, alkyl or heterosubstituted phenyl. In one preferred embodiment, R₁₆ is hydrogen and R₁₅ is O₁₋₈ alkyl, phenyl, C₁₋₈ alkyl substituted phenyl, or substituted phenyl. In another embodiment, R₁₆ is hydrocarbyl or substituted hydrocarbyl; for example, in this embodiment, R₁₆ may be hydrocarbyl or substituted hydrocarbyl and R₁₅ may be alkyl, alkenyl, alkynyl, phenyl, alkyl or heterosubstituted phenyl.

In a preferred embodiment, the carbene precursor is a diazosulfone, R₁₆ is hydrogen, and R₁₅ is toluene. In this embodiment, the diazosulfone corresponds to the following structure, also referred to herein as N₂CHTs:

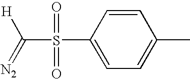

In accordance with one embodiment of the present invention, an olefin is converted to a cyclopropane as illustrated in Reaction Scheme A:

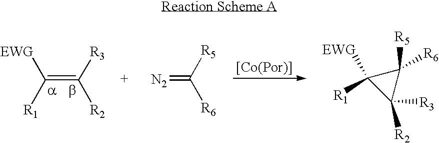

wherein [Co(Por)] is a cobalt porphyrin complex; R₁ is hydrogen, hydrocarbyl, substituted hydrocarbyl, or heterocyclo; R₂, and R₃ are independently hydrogen, hydrocarbyl, substituted hydrocarbyl, heterocyclo, or EWG₂; R₅ and R₆ are independently hydrogen, hydrocarbyl, substituted hydrocarbyl, heterocyclo, nitro, or sulfonyl; and EWG and EWG₂ are independently an electron-withdrawing group. In one embodiment, R₁, R₂, the α-carbon, and the β-carbon form a carbocyclic or heterocyclic ring. In another embodiment, R₂, R₃, and the β-carbon form a carbocyclic or heterocyclic ring. In another embodiment, R₁, R₃, the α-carbon, and the β-carbon form a carbocyclic or heterocyclic ring. In a preferred embodiment, one of R₅ and R₆ is hydrogen and the other is carbonyl. In another preferred embodiment, one of R₅ and R₆ is hydrogen and the other is an ester (—C(O)OR wherein R is hydrocarbyl, substituted hydrocarbyl, or heterocyclo). In another preferred embodiment, one of R₅ and R₆ is hydrogen and the other is a sulfonyl moiety. In another preferred embodiment, one of R₅ and R₆ is a carbonyl and the other is a nitro compound.

In one preferred embodiment, an olefin is converted to a cyclopropane as illustrated in Reaction Scheme 1.

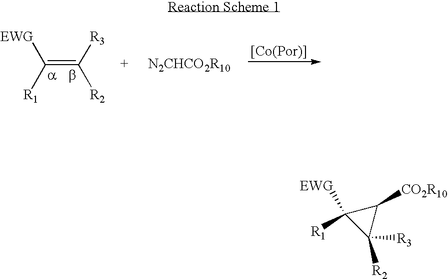

wherein [Co(Por)] is a cobalt porphyrin complex; R₁ is hydrogen, hydrocarbyl, substituted hydrocarbyl, or heterocyclo; R₂, and R₃ are independently hydrogen, hydrocarbyl, substituted hydrocarbyl, heterocyclo, or EWG₂; R₁₀ is hydrocarbyl, substituted hydrocarbyl, or heterocyclo; and EWG and EWG₂ are independently an electron-withdrawing group. In one embodiment, R₁, R₂, the α-carbon, and the β-carbon form a carbocyclic or heterocyclic ring. In another embodiment, R₂, R₃, and the β-carbon form a carbocyclic or heterocyclic ring. In another embodiment, R₁, R₃, the α-carbon, and the β-carbon form a carbocyclic or heterocyclic ring.

In another preferred embodiment, an olefin is converted to a cyclopropane as illustrated in Reaction Scheme 1b:

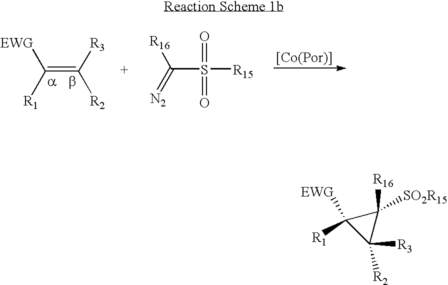

wherein [Co(Por)] is a cobalt porphyrin complex; R₁ is hydrogen, hydrocarbyl, substituted hydrocarbyl, or heterocyclo; R₂, and R₃ are independently hydrogen, hydrocarbyl, substituted hydrocarbyl, heterocyclo, or EWG₂; R₁₆ is hydrogen; R₁₅ is hydrogen, hydrocarbyl, substituted hydrocarbyl, or heterocyclo; and EWG and EWG₂ are independently an electron-withdrawing group. In one embodiment, R₁, R₂, the α-carbon, and the β-carbon form a carbocyclic or heterocyclic ring. In another embodiment, R₂, R₃, and the β-carbon form a carbocyclic or heterocyclic ring. In another embodiment, R₁, R₃, the α-carbon, and the β-carbon form a carbocyclic or heterocyclic ring. In another preferred embodiment, an olefin is converted to a cyclopropane as illustrated in Reaction Scheme 1c:

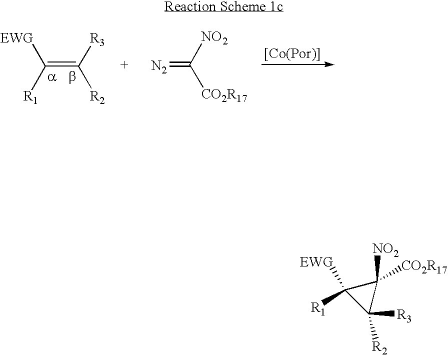

wherein [Co(Por)] is a cobalt porphyrin complex; R₁ is hydrogen, hydrocarbyl, substituted hydrocarbyl, or heterocyclo; R₂, and R₃ are independently hydrogen, hydrocarbyl, substituted hydrocarbyl, heterocyclo, or EWG₂; R₁₇ is hydrogen, hydrocarbyl, substituted hydrocarbyl, or heterocyclo; and EWG and EWG₂ are independently an electron-withdrawing group. In one embodiment, R₁, R₂, the α-carbon, and the β-carbon form a carbocyclic or heterocyclic ring. In another embodiment, R₂, R₃, and the β-carbon form a carbocyclic or heterocyclic ring. In another embodiment, R₁, R₃, the α-carbon, and the β-carbon form a carbocyclic or heterocyclic ring.

In accordance with one preferred embodiment, the electron withdrawing group, EWG, is a carbonyl or nitrile group and reaction proceeds as depicted in Reaction Schemes 2 and 3, respectively:

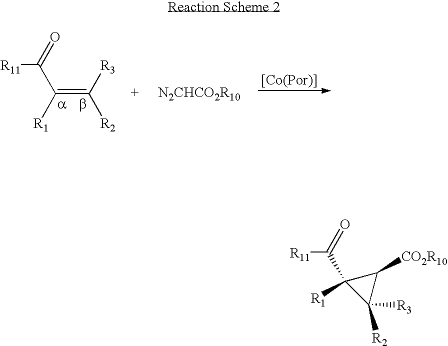

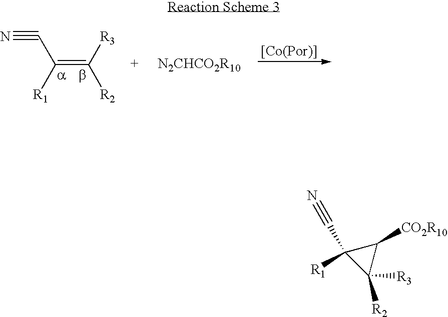

wherein [Co(Por)] is a cobalt porphyrin complex; R₁ is hydrogen, hydrocarbyl, substituted hydrocarbyl, or heterocyclo; R₂, and R₃ are independently hydrogen, hydrocarbyl, substituted hydrocarbyl, heterocyclo, or EWG₂; R₁₀ is hydrocarbyl, substituted hydrocarbyl, or heterocyclo; R₁₁ is hydrogen, hydrocarbyl, substituted hydrocarbyl, heterocyclo, —NRₐR_{b}, —ORₐ, or —OC(O)OC(O)Rₐ; Rₐ and R_{b} are independently hydrogen, hydrocarbyl, substituted hydrocarbyl or heterocyclo; and EWG and EWG₂ are independently an electron-withdrawing group. In one such embodiment in which the cyclopropanation reaction proceeds as set forth as depicted in Reaction Scheme 2 or 3, R₁ is hydrogen. In another embodiment in which the cyclopropanation reaction proceeds as depicted in Reaction Scheme 2 or 3, R₁ is hydrogen and R₂ and R₃ are independently hydrogen, hydrocarbyl, substituted hydrocarbyl or heterocyclo. In another embodiment in which the cyclopropanation reaction proceeds as depicted in Reaction Scheme 2 or 3, R₁ is hydrogen and one of R₂ and R₃ is hydrogen. In another embodiment in which the cyclopropanation reaction proceeds as depicted in Reaction Scheme 2 or 3, R₁ is hydrogen, one of R₂ and R₃ is hydrogen, and the other of R₂ and R₃ is hydrocarbyl, substituted hydrocarbyl, or heterocyclo. In a further embodiment, R₁₀ may be alkyl, typically lower alkyl.

In accordance with one embodiment, each of the ethylenic carbons possesses an electron withdrawing group and the cyclopropanation reaction proceeds as depicted in Reaction Scheme 4 or 5:

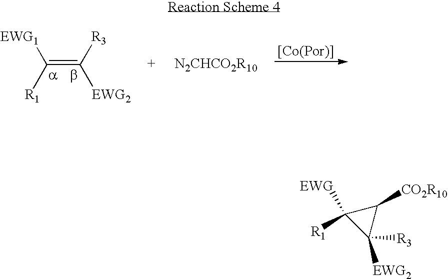

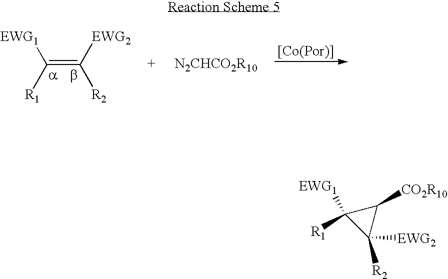

wherein [Co(Por)] is a cobalt porphyrin complex; R₁, R₂, and R₃ are independently hydrogen, hydrocarbyl, substituted hydrocarbyl, heterocyclo; R₁₀ is hydrocarbyl, substituted hydrocarbyl, or heterocyclo; and EWG₁ and EWG₂ are independently an electron-withdrawing group. In one such embodiment in which the cyclopropanation reaction proceeds as set forth as depicted in Reaction Scheme 4 or 5, R₁ is hydrogen. In another embodiment in which the cyclopropanation reaction proceeds as set forth as depicted in Reaction Scheme 4 or 5, R₁ is hydrogen and R₂ and R₃ are hydrogen, hydrocarbyl, substituted hydrocarbyl or heterocyclo. In another embodiment in which the cyclopropanation reaction proceeds as depicted in Reaction Scheme 4 or 5, R₁ is hydrogen and R₂ or R₃ is hydrogen. In another embodiment in which the cyclopropanation reaction proceeds as depicted in Reaction Scheme 4 or 5, R₁ is hydrogen, and R₂ or R₃ is hydrocarbyl, substituted hydrocarbyl, or heterocyclo. In a further embodiment, R₁₀ may be alkyl, typically lower alkyl.

The enantioselectivity and diastereoselectivity can be influenced, at least in part, by selection of the cobalt porphyrin complex. Similarly, stereoselectivity of the reaction may also be influenced by the selection of chiral porphyrin ligands with desired electronic, steric, and chiral environments. Accordingly, the catalytic system of the present invention may advantageously be used to control stereoselectivity.

In one embodiment, the metal of the metal porphyrin complex is a transition metal. Thus, for example, the metal may be any of the 30 metals in the 3d, 4d, and 5d transition metal series of the Periodic Table of the Elements, including the 3d series that includes Sc, Ti, V, Cr, Mn, Fe, Co, Ni, Cu, and Zn; the 4d series that includes Y, Zr, Nb, Mo, Tc, Ru, Rh, Pd, Ag and Cd; and the 5d series that includes Lu, Hf, Ta, W, Re, Os, Ir, Pt, Au and Hg. In some embodiments, M is a transition metal from the 3d series. In some embodiments, M is selected from the group consisting of Co, Zn, Fe, Ru, Mn, and Ni. In some embodiments, M is selected from the group consisting of Co, Fe, and Ru. In some embodiments, M is Co.

The porphyrin with which cobalt is complexed may be any of a wide range of porphyrins known in the art. Exemplary porphyrins are described in U.S. Patent Publication Nos. 2005/0124596 and 2006/0030718 and U.S. Pat. No. 6,951,934 (each of which is incorporated herein by reference, in its entirety). Exemplary porphyrins are also described in Chen et al., Bromoporphyrins as Versatile Synthons for Modular Construction of Chiral Porphyrins: Cobalt-Catalyzed Highly Enantioselective and Diastereoselective Cyclopropanation (J. Am. Chem. Soc. 2004), which is incorporated herein by reference in its entirety.

In one embodiment, the cobalt porphyrin complex is a cobalt (II) porphyrin complex. In one particularly preferred embodiment, the cobalt porphyrin complex is a D₂-symmetric chiral porphyrin complex corresponding to the following structure

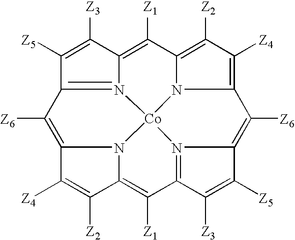

wherein each Z₁, Z₂, Z₃, Z₄, Z₅ and Z₆ are each independently selected from the group consisting of X, H, alkyl, substituted alkyls, arylalkyls, aryls and substituted aryls; and X is selected from the group consisting of halogen, trifluoromethanesulfonate (OTf), haloaryl and haloalkyl. In a preferred embodiment, Z₂, Z₃, Z₄ and Z₅ are hydrogen, Z₁ is a substituted phenyl, and Z₆ is substituted phenyl, and Z₁ and Z₆ are different. In one particularly preferred embodiment, Z₂, Z₃, Z₄ and Z₅ are hydrogen, Z₁ is substituted phenyl, and Z₆ is substituted phenyl and Z₁ and Z₆ are different and the porphyrin is a chiral porphyrin. In one even further preferred embodiment, Z₂, Z₃, Z₄ and Z₅ are hydrogen, Z₁ is substituted phenyl, and Z₆ is substituted phenyl and Z₁ and Z₆ are different and the porphyrin has D₂-symmetry.

Exemplary cobalt (II) porphyrins include the following:

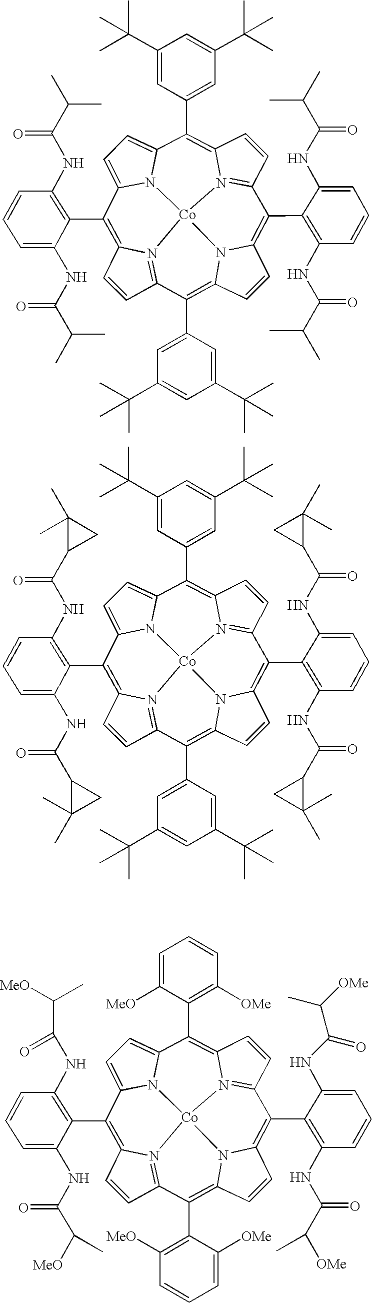

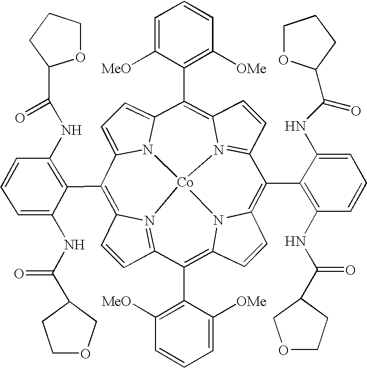

In an embodiment, the cobalt porphyrin complex corresponds to [Co(P6)]:

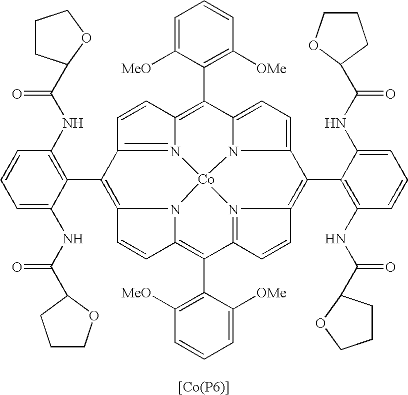

In an embodiment, the cobalt porphyrin complex corresponds to [Co(P1)]:

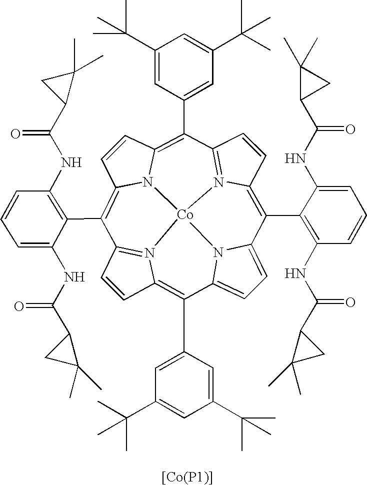

The cyclopropanation reaction could be carried out efficiently at room temperature in a one-pot fashion with olefins as limiting reagents and would not require the slow-addition of ester reagents. Additionally, the cyclopropanation reaction may be operated with relatively low catalyst loading, in a solvent such as toluene, chlorobenzene, tetrahydrofuran (THF), dichloromethane, or acetonitrile. The enantioselectivity and diastereoselectivity can be influenced, at least in part, by the selection of the solvent. In a preferred embodiment, the solvent is chlorobenzene, which was found to give the desired cyclopropane in the highest yield and with the best enantioselectivity as well as diastereoselectivity.

One aspect of the present invention is a general and efficient catalytic system for asymmetric cyclopropanation of electron-deficient olefins. Building on our previous work on Co-based asymmetric cyclopropanation, (Huang, L.; Chen, Y.; Gao, G.-Y.; Zhang, X. P. J. Org. Chem. 2003, 68, 8179; Chen, Y.; Fields, K. B.; Zhang, X. P. J. Am. Chem. Soc. 2004, 126, 14718; and Chen, Y.; Zhang, X. P. J. Org. Chem. 2007, 72, 5931), the Co(II) complex of the D₂-symmetric chiral porphyrin [Co(1)] (Formula 4b) was found to cyclopropanate a wide range of α,β-unsaturated carbonyl compounds and nitriles (Reaction Scheme B), forming the corresponding electrophilic cyclopropane derivatives in high yields and selectivities. Furthermore, the [Co(1)]-based catalytic process could be operated efficiently at room temperature in a one-pot fashion with olefins as limiting reagents and would not require the slow-addition of diazo reagents.

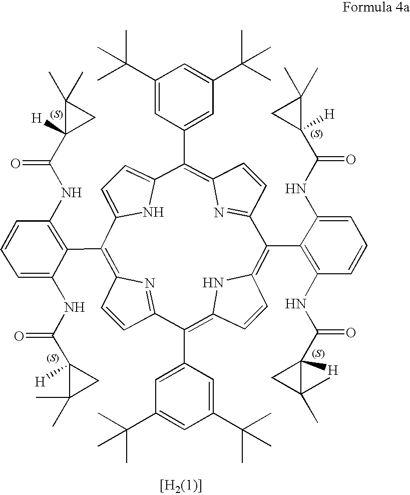

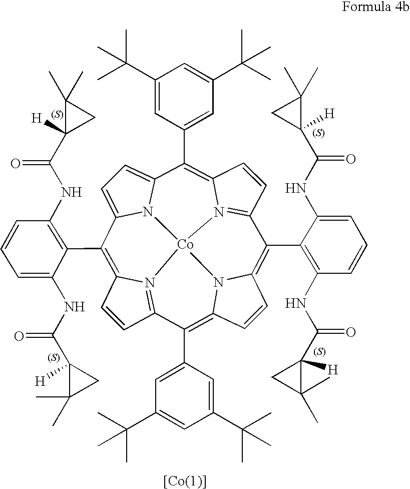

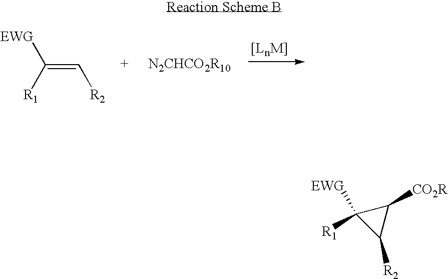

Having described the invention in detail, it will be apparent that modifications and variations are possible without departing the scope of the invention defined in the appended claims. Furthermore, it should be appreciated that all examples in the present disclosure are provided as non-limiting examples.

### EXAMPLES

Previous studies on asymmetric cyclopropanation of styrene derivatives revealed that a [Co(1)]-based system seemed insensitive to substrate electronics. (Huang et al, J. Org. Chem. 2003, 68, 8179; Chen et al., J. Am. Chem. Soc. 2004, 126, 14718; and Chen Y., Zhang, X. P., J. Org. Chem. 2007, 72, 5931.) Even the extremely electron-deficient pentafluorostyrene could be cyclopropanated. (Chen Y., Zhang, X. P., J. Org. Chem. 2007, 72, 5931.) This result prompted us to evaluate the catalytic reactivity of [Co(1)] toward more challenging substrates such as electron-deficient non-styrene olefins (Table 1). Under the one-pot protocol where olefins are the limiting reagent, using 1 mol % [Co(1)] in the presence of 0.5 equivalents of DMAP could effectively cyclopropanate both acrylates and methacrylates with EDA or tert-butyl diazoacetate (t-BDA) at room temperature in toluene, forming the corresponding 1,2-cyclopropanediesters in good yields and high diastereo- as well as enantio-selectivities (Table 1, entries 1-5). Under the same conditions, acrylamide as well as its mono- and di-substituted derivatives were also suitable substrates, providing the corresponding 1,2-cyclopropaneamidoesters with good to high yields and excellent selectivities (Table 1, entries 6-10). The amido functional groups were well tolerated; no N—H insertion products were observed. Alkenes bearing carbonyl and cyano groups such as acrylketones and acrylonitriles were fully compatible with the catalytic system as well. In most of the cases, the resulting 1,2-cyclopropaneketoesters (Table 1, entries 11-15) and 1,2-cyclopropane cyanoesters (Table 1, entries 16-19) could be synthesized in high yields and high selectivities. As the best example, cyclopropanation of 1-octen-3-one with t-BDA resulted in the formation of the desired trans-1,2-cyclopropaneketoester in 94% yield, 98% de, and 96% ee (Table 1, entry 14). Diethyl maleate could also be successfully cyclopropanated to produce the 1,2,3-cyclopropanetriester solely as the α,α,β-isomer, albeit in a lower yield (Table 1, entry 20).

While most of the substrates gave high yields and selectivities, the yields of several reactions were still moderate (Table 1). To further improve the catalytic process without sacrificing its attractive practicality, several common solvents in addition to toluene were evaluated for the cyclopropanation of ethyl acrylate with t-BDA under the same conditions. Among the solvents tested (Table 2), chlorobenzene was found to be the best solvent, giving the desired cyclopropane in the highest yield and with the best enantioselectivity as well as diastereoselectivity. As a result, several lower-yielding reactions were repeated in chlorobenzene. Dramatic improvements in yield were obtained while maintaining high diastereo- and enantio-selectivities (Table 1, entries 1A-3A, 5A-7A, 10A, 15A, 18A, and 20A).

As demonstrated by the results reported in Table 1, [Co(1)] is an effective catalyst for asymmetric cyclopropanation of various electron-deficient olefins under mild conditions, forming synthetically valuable electrophilic cyclopropane derivatives in high yields and high stereoselectivities. Together with its high reactivity and selectivity toward styrene derivatives shown previously, [Co(1)] may be considered one of the most selective catalysts for asymmetric cyclopropanation of both electron-sufficient and electron-deficient olefins with diazoacetates.(Lebel et al., Chem. Rev. 2003, 103, 977; Davies H. M. L., Antoulinakis E., Org. React. 2001, 57, 1; Doyle M. P., Forbes D. C., Chem. Rev. 1998, 98, 911; Padwa A., Krumpe K. E., Tetrahedron 1992, 48, 5385-5453; Pietruszka J., Chem. Rev. 2003, 103, 1051; Wessjohann et al, Chem. Rev. 2003, 103, 1625; Donaldson W. A., Tetrahedron 2001, 57, 8589; Salaun J., Chem. Rev. 1989, 89, 1247; Fritschi et al, Agnew. Chem., Int. Ed. Engl. 1986, 25, 1005; Evans et al, J. Am. Chem. Soc. 1991, 113, 726; Lo et al, J. Am. Chem. Soc. 1998, 120, 10270; Maxwell et al, Organometallics 1992, 11, 645; Doyle et al, J. Am. Chem. Soc. 1993, 115, 9968; Davies et al., J. Am. Chem. Soc. 1996, 118, 6897; Nishiyama et al, J. Am. Chem. Soc. 1994, 116, 2223; Che et al, J. Am. Chem. Soc. 2001, 123, 4119.)

These results suggest that the catalytic intermediate of the current Co(II)-based system may have different reactivity characteristics from the previously reported either Cu(I)— or Rh(II)₂-based systems.

TABLE 1
Diastereoselective and Enantioselective Cyclopropanation of Electron-
Deficient Olefins Catalyzed by [CO(1)].^{a}

| | | | | yield | | ee |
|---|---|---|---|---|---|---|
| entry | alkene | diazo | product | (%)^{c} | t:c^{d} | (%)^{e} |
| 1 1A^{b} | 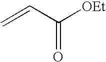 | EDA | 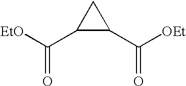 | 78 95 | 98:02 97:03 | 80^{g} 81^{g} |
| 2 2A^{b} | 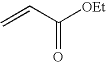 | t-BDA | 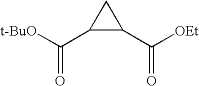 | 72 92 | 99:01 99:01 | 90 91 |
| 3 3A^{b} | 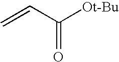 | t-BDA | 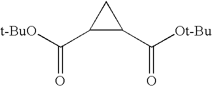 | 62 88 | 98:02 97:03 | 84 80 |
| 4 | 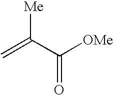 | EDA | 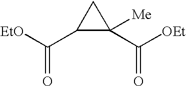 | 73 | 95:05 | 61 |
| 5 5A^{b} | 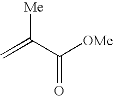 | t-BDA | 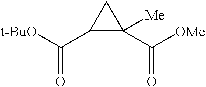 | 62 90 | 93:07 93.07 | 84 83 |
| 6 6A^{b} | 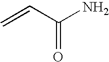 | EDA | 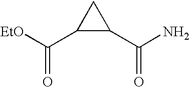 | 51 81 | 99:01 99:01 | 88 90 |
| 7 7A^{b} | 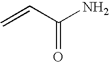 | t-BDA | 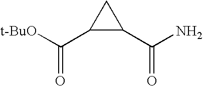 | 66 77 | 99:01 99:01 | 97 97 |
| 8 | 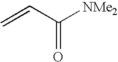 | EDA | 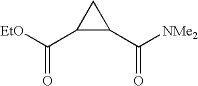 | 85 | 99:01 | 77 |
| 9 | 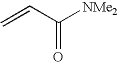 | t-BDA | 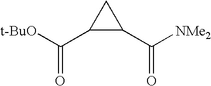 | 86 | 99:01 | 96 |
| 10 10A^{b} | 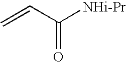 | t-BDA | 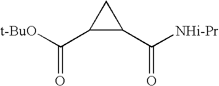 | 44 96 | 99:01 99:01 | 97 96 |
| 11 | 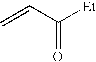 | EDA | 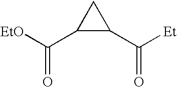 | 89 | 96:04 | 80 |
| 12 | 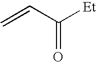 | t-BDA | 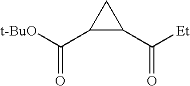 | 81 | 99:01 | 94 |
| 13 | 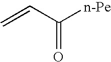 | EDA | 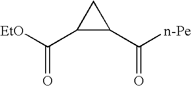 | 92 | 98:02 | 79 |
| 14 | 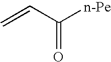 | t-BDA | 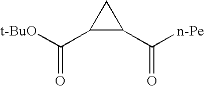 | 94 | 99:01 | 96 |
| 15 15A^{b} | 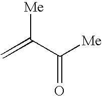 | t-BDA | 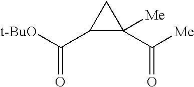 | 40 84 | 98:02 97:03 | 90 87 |
| 16 | 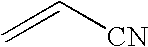 | EDA | 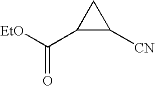 | 83 | 72:28 | 73 |
| 17 | 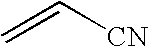 | t-BDA | 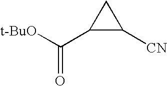 | 83 | 76.24 | 93 |
| 18 18A^{b} | 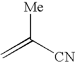 | EDA | 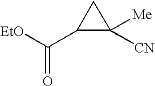 | 77 93 | 69:31 69:31 | 84 81 |
| 19 | 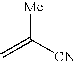 | t-BDA | 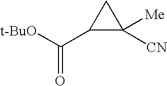 | 87 | 62:38 | 95 |
| 20 20A^{b} | 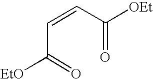 | EDA | 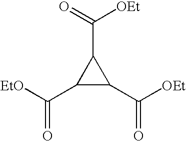 | 37 94 | >99:1^{t} >99:1^{t} | — — |

^{a}Performed in toluene at RT for 20 h using 1 mol % [Co(1)] under N₂ with 1.0 equiv of alkene and 1.2 equiv of EDA or t-BDA in the presence of 0.5 equiv of DMAP. [alkene] = 0.25 M.
^{b}Performed in chlorobenzene.
^{c}Isolated yields.
^{d}Determined by GC.
^{e}Determined by GC or HPLC on chiral stationary phases.
^{f}Only the α,α,β-isomer was observed.
^{g}(−)-[1R,2R] absolute configuration determined by optical rotation.

TABLE 2
Solvent Effect in [Co(1)]-Catalyzed Diastereoselective and
Enantioselective Cyclopropanation of Electron-Deficient Olefins.^{a}

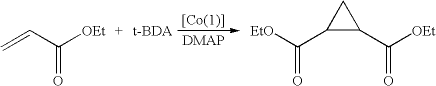

| entry | solvent | yield(%)^{b} | trans:cis^{c} | ee(%)^{d} |
|---|---|---|---|---|
| 1 | MeC₆H₅ | 72 | 99:01 | 90 |
| 2 | ClC₆H₅ | 92 | 99:01 | 91 |
| 3 | THF | 29 | 88:12 | 76 |
| 4 | CH₂Cl₂ | 61 | 99:01 | 85 |
| 5 | CH₃CN | 58 | 96:04 | 84 |

^{a}Performed at room temperature for 20 h using 1 mol % [Co(1)] under N₂ with 1.0 equiv of alkene and 1.2 equiv of t-BDA in the presence of 0.5 equiv of DMAP. [alkene] = 0.25 M.
^{b}Isolated yields.
^{c}The trans:cis ratios were determined by GC.
^{d}The ee of trans isomer was determined by chiral GC or chiral HPLC.

The cobalt porphyrin complex [Co(P6)] was shown to be a general catalyst for a range of aromatic and electron-deficient terminal olefins and with different diazoarylsulfones (Table 3). Electron-deficient olefins, such as α,β-unsaturated esters (entries 1-3), ketones (entry 4), and nitriles (entry 5), could be effectively cyclopropanated with N₂CHTs by [Co(P6)]. Except for the case of an α,β-unsaturated nitrile (entry 14), all the corresponding cyclopropyl sulfones were formed in high enantioselectivity and excellent trans diastereoselectivity (Table 3). Cyclopropyl sulfones that are most enantiomerically pure (>98% ee) were obtained through a simple recrystallization procedure due to the high crystalline nature of the class of compounds, as exemplified in the methyl vinyl ketone reaction (entry 4).

TABLE 3
[Co(P6)]-Catalyzed Diastereo- and Enantioselective
Cyclopropanation of Different Alkenes with Various Diazosulfones^{a}

| Entry | Olefin | Cyclopropane | Y (%)^{b} | t:c^{c} | ee(%)^{d} | [α]^{e} |
|---|---|---|---|---|---|---|
| 1^{h} | 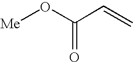 | 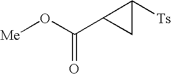 | 96 | 94:06 | 89 | (−) |
| 2ⁱ | 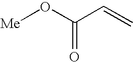 | 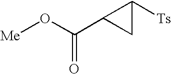 | 64 | >99:01 | 97 | (−) |
| 3^{h} | 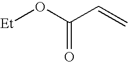 | 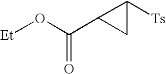 | 72 | >99:01 | 90 | (−) |
| 4^{h} | 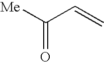 | 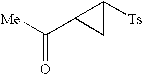 | 93 (81)^{j} | >99:01 (>99:01)^{j} | 89 (98) | (−) |
| 5^{h} | 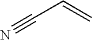 | 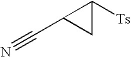 | 81 | 79:21 | 61 | (−) |

^{a}Performed in CH₂Cl₂ at room temperature for 24 hours using 1 mol % of [Co(Por)] under N₂ with 1.0 equivalent of styrene and 1.2 equivalent of N₂CHTs; [styrene] = 0.25 M.
^{b}Isolated yields.
^{c}The cis:trans ratio determined by NMR.
^{d}The trans isomer ee was determined by chiral HPLC.
^{e}Sign of optical rotation.
^{h}In ClC₆H₅ at room temperature for 24 hours using 2 mol % of [Co(P6)].
^{j}After one recrystallization.

The substrate scope of the [Co(P1)]-based catalytic system was then examined. As summarized in Table 4, the catalytic process could be successfully applied for different kinds of alkene substrates with various nitro diazoacetate derivatives. Using 1,2-dichloroethane as solvent, electron-deficient olefins such as α,β-unsaturated esters and amides, which represent a class of difficult substrates, could be cyclopropanated as well, but with diminished diastereoselectivity (Table 4).

TABLE 4
[Co(P1)]-Catalyzed Diastereo- and Enantioselective Cyclopropanation of
Different Alkenes with α-Nitro-Diazoacetates.^{a}

| | | | yield | | | |
|---|---|---|---|---|---|---|
| Entry | Cyclopropane | R | (%)^{b} | cis:trans^{c} | ee(%)^{d} | [α]^{e} |
| 1^{hj} | 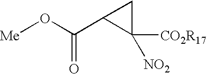 | Et | 42 | 53:47 | 88 | (−) |
| 2^{hj} | 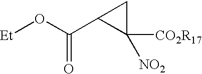 | Et | 62 | 56:44 | 88 | (−) |
| 3^{hj} | 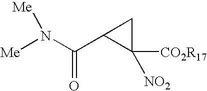 | Et | 92 | 63:37 | 75 | (−) |

^{a}Performed in n-hexane at RT for 24 h using 1 mol % [Co(P1)] under N₂ with 1.0 equiv of alkene and 1.2 equiv of NDA. [alkene] = 0.25 M.
^{b}Isolated yields.
^{c}Determined by NMR.
^{d}Cis ee determined by chiral HPLC.
^{e}Sign of optical rotation.
^{h}5 mol %.
^{j}In C₂H₄Cl₂.

All reactions were carried out under a nitrogen atmosphere in oven-dried glassware following standard Schlenk techniques. Proton and carbon nuclear magnetic resonance spectra (¹H NMR and ¹³C NMR) were recorded on a Varian Mercury 300 spectrometer and referenced with respect to internal TMS standard or residual solvent. HPLC measurements were carried out on a Hewlett-Packard HP1100 system with Whelk-O1 or Chiralcel OD-H column. GC-MS analysis was performed on a Hewlett-Packard G 1800B GCD system equipped with a CP-Chirasil-Dex CB or a Chiraldex G-TA column. Infrared spectra were obtained by using a Bomen B100 Series FT-IR spectrometer. HRMS data was obtained on an Agilent 1100 LC/MS ESI/TOF mass spectrometer with electrospray ionization. Optical rotation was performed on a Rudolph Research Analytical Autopol IV polarimeter (λ=365 nm) using a 0.8-mL cell with path length of 1-dm.

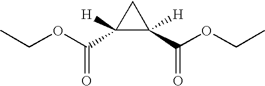

(−)-(1R,2R)-diethyl 1,2-cyclopropanedicarboxylate (Csuk R., von Scholz Y., Tetrahedron 1994, 50, 10431; Jeromin et al., Ger. Offen. 2006.) (Entry 1, Table 1) was synthesized from ethyl acrylate with EDA. trans-isomer: [α]²⁷₃₆₅=−452.1 (c=0.42, CHCl₃). ¹H NMR (300 MHz, CDCl₃): δ4.15 (q, J=7.2 Hz, 4H), 2.13-2.18 (m, 2H), 1.40-1.45 (m, 2H), 1.28 (t, J=7.2 Hz, 6H). ¹³C NMR (75 MHz, CDCl₃): δ171.8, 61.0, 22.3, 15.3, 14.1. IR (film, cm⁻¹): 1728 (C═O). HRMS (ESI): Calcd. for C₉H₁₅O₄ ([M+H]⁺) m/z 187.0970, Found 187.0964. GC analysis: Chiraldex G-TA (Temp program: initial temp=50° C., 2.00° C./min, final temp=180° C., final time=10.00 min) trans-isomer: tₘᵢₙₒᵣ=30.46 min, tₘₐⱼₒᵣ=30.74 min.

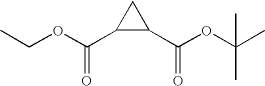

tert-Butyl ethyl 1,2-cyclopropanedicarboxylate (Bonavent et al., Bull. Soc. Chim. Fr. 1964, 10, 2462.) (Entry 2, Table 1) was synthesized from tert-butyl acrylate with EDA or from ethyl acrylate with t-BDA. trans-isomer: ¹H NMR (300 MHz, CDCl₃): δ4.15 (q, J=7.2 Hz, 2H), 2.06-2.11 (m, 2H), 1.45 (s, 9H), 1.34-1.39 (m, 2H), 1.28 (t, J=7.2 Hz, 3H). ¹³C NMR (75 MHz, CDCl₃): δ172.0, 170.9, 81.2, 61.0, 28.0, 23.3, 22.0, 15.2, 14.1. IR (film, cm⁻¹): 1725 (C═O). HRMS (ESI): Calcd. for C₁₁H₂₂O₄N ([M+NH₄]⁺) m/z 232.1549, Found 232.1545. GC analysis: CP-Chirasil-Dex CB (Temp program: initial temp=50° C., Rate₁: 10.00° C./min, max temp=100° C.; Rate₂: 2.00° C./min, max temp=140° C.; Rate₃: 10.00° C./min, max temp=200° C.; final time=0.00 min) trans-isomer: tₘᵢₙₒᵣ=21.18 min, tₘₐⱼₒᵣ=21.36 min.

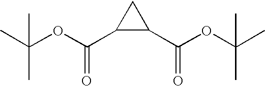

Di-tert-butyl 1,2-cyclopropanedicarboxylate (Artaud et al., Acad. Sci., Ser. IIc: Chim. 1976, 283, 503.) (Entry 3, Table 1) was synthesized from tert-butyl acrylate with t-BDA. trans-isomer: ¹H NMR (300 MHz, CDCl₃): δ1.98-2.02 (m, 2H), 1.45 (s, 18H), 1.26-1.31 (m, 2H). ¹³C NMR (75 MHz, CDCl₃): δ171.1, 81.0, 28.0, 23.1, 15.2. IR (film, cm⁻¹): 1724 (C═O). HRMS (ESI): Calcd. for C₁₃H₂₆O₄N ([M+NH₄]⁺) m/z 260.1862, Found 260.1856. GC analysis: CP-Chirasil-Dex CB (Temp program: initial temp=50° C., 10.00° C./min, final temp=200° C., final time=10.00 min) trans-isomer: tₘᵢₙₒᵣ=14.54 min, tₘₐⱼₒᵣ=14.59 min.

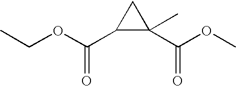

Ethyl methyl 1-methyl-1,2-cyclopropanedicarboxylate (Doyle et al., J. Org. Chem. 1982, 47, 4059.) (Entry 4, Table 1) was synthesized from methyl methacrylate with EDA. trans-isomer: ¹H NMR (300 MHz, CDCl₃): δ4.17 (q, J=7.2 Hz, 2H), 3.70 (s, 3H), 2.30-2.36 (m, 1H), 1.55-1.59 (m, 1H), 1.40 (s, 3H), 1.31-1.34 (m, 1H), 1.28 (t, J=7.2 Hz, 3H). ¹³C NMR (75 MHz, CDCl₃): δ174.0, 170.3, 60.9, 52.3, 27.9, 26.8, 20.9, 14.2, 13.0. IR (film, cm⁻¹): 1727 (C═O). HRMS (ESI): Calcd. for C₉H₁₈O₄N ([M+NH₄]⁺) m/z 204.1236, Found 240.1229. GC analysis: CP-Chirasil-Dex CB (Temp program: initial temp=50° C., Rate₁: 3.00° C./min, max temp=100° C.; Rate₂: 2.00° C./min, max temp=130° C.; Rate₃: 10.00° C./min, max temp=200° C.; final time=5.00 min) trans-isomer: tₘᵢₙₒᵣ=25.36 min, tₘₐⱼₒᵣ=25.47 min.

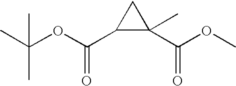

tert-Butyl methyl 1-methyl-1,2-cyclopropanedicarboxylate (Entry 5, Table 1) was synthesized from methyl methacrylate with t-BDA. trans-isomer: ¹H NMR (300 MHz, CDCl₃): δ3.69 (s, 3H), 2.23-2.28 (m, 1H), 1.49-1.52 (m, 1H), 1.46 (s, 9H), 1.39 (s, 3H), 1.24-1.27 (m, 1H). ¹³C NMR (75 MHz, CDCl₃): δ174.2, 169.5, 81.1, 52.3, 28.3, 28.1, 26.5, 20.6, 12.9. IR (film, cm⁻¹): 1725 (C═O). HRMS (ESI): Calcd. for C₁₁H₂₂O₄N ([M+NH₄]⁺) m/z 232.1549, Found 232.1541. HPLC analysis: Whelk-O1 (98% hexanes: 2% isopropanol, 1.0 mL/min) trans-isomer: tₘₐⱼₒᵣ=7.01 min, tₘᵢₙₒᵣ=7.57 min.

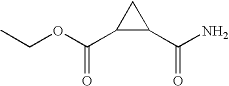

Ethyl 2-aminocarbonyl-cyclopropanecarboxylate (Kennewell et al., J. Chem. Soc., Perkin Trans. 1, 1982, 11, 2563.) (Entry 6, Table 1) was synthesized from acrylamide with EDA. trans-isomer: ¹H NMR (300 MHz, CDCl₃): δ5.80 (br, 2H), 4.15 (q, J=7.2 Hz, 2H), 2.14-2.20 (m, 1H), 1.99-2.05 (m, 1H), 1.43-1.49 (m, 1H), 1.33-1.38 (m, 1H), 1.28 (t, J=7.2 Hz, 3H). ¹³C NMR (75 MHz, CDCl₃): δ172.5, 61.1, 23.4, 21.9, 15.0, 14.2. IR (film, cm⁻¹): 3202-3420 (NH), 1721 (C═O), 1669 (C═O), 1622 (C═O). HRMS (ESI): Calcd. for C₇H₁₂NO₃ ([M+H]⁺) m/z 158.0817, Found 158.0813. GC analysis: CP-Chirasil-Dex CB (Temp program: initial temp=50° C., 10.00° C./min, final temp=200° C., final time=10.00 min) trans-isomer: tₘₐⱼₒᵣ=17.36 min, tₘᵢₙₒᵣ=17.42 min.

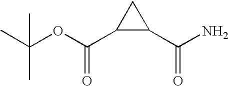

tert-Butyl 2-aminocarbonyl-cyclopropanecarboxylate (Entry 7, Table 1) was synthesized from acrylamide with t-BDA. trans-isomer: ¹H NMR (300 MHz, CDCl₃): δ6.08 (br, 2H), 2.04-2.10 (m, 1H), 1.93-1.99 (m, 1H), 1.45 (s, 9H), 1.32-1.41 (m, 1H), 1.26-1.30 (m, 1H). ¹³C NMR (75 MHz, CDCl₃): δ173.2, 171.7, 81.2, 28.0, 23.1, 22.9, 14.8. IR (film, cm⁻¹): 3205-3421 (NH), 1717 (C═O), 1671 (C═O), 1623 (C═O). HRMS (ESI): Calcd. for C₉H₁₉N₂O₃ ([M+NH₄]⁺) m/z 203.1396, Found 203.1389. GC analysis: CP-Chirasil-Dex CB (Temp program: initial temp=50° C., 10.00° C./min, final temp=200° C., final time=10.00 min) trans-isomer: tₘᵢₙₒᵣ=17.59 min, tₘₐⱼₒᵣ=17.76 min.

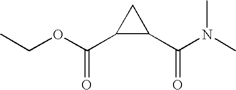

Ethyl 2-dimethylaminocarbonyl-cyclopropanecarboxylate (Entry 8, Table 1) was synthesized from N,N-dimethylacrylamide with EDA. trans-isomer: ¹H NMR (300 MHz, CDCl₃): δ4.15 (q, J=7.2 Hz, 2H), 3.18 (s, 3H), 2.97 (s, 3H), 2.29-2.36 (m, 1H), 2.14-2.20 (m, 1H), 1.40-1.48 (m, 1H), 1.31-1.37 (m, 1H), 1.27 (t, J=7.2 Hz, 3H). ¹³C NMR (75 MHz, CDCl₃): δ172.8, 170.0, 60.7, 37.1, 35.7, 21.6, 20.8, 15.1, 14.0. IR (film, cm⁻¹): 3490 (NH), 1725 (C═O), 1642 (C═O). HRMS (ESI): Calcd. for C₉H₁₅NO₃Na ([M+Na]⁺) m/z 208.0950, Found 208.0942. HPLC analysis: Chiralcel OD-H (90% hexanes: 10% isopropanol, 1.0 mL/min) trans-isomer: tₘₐⱼₒᵣ=10.67 min, tₘᵢₙₒᵣ=11.78 min.

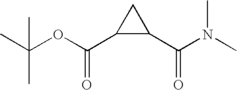

tert-Butyl 2-dimethylaminocarbonyl-cyclopropanecarboxylate (Entry 9, Table 1) was synthesized from N,N-dimethylacrylamide with t-BDA. trans-isomer: ¹H NMR (300 MHz, CDCl₃): δ3.17 (s, 3H), 2.97 (s, 3H), 2.22-2.28 (m, 1H), 2.07-2.13 (m, 1H), 1.45 (s, 9H), 1.35-1.40 (m, 1H), 1.26-1.32 (m, 1H). ¹³C NMR (75 MHz, CDCl₃): δ 172.1, 170.4, 80.9, 37.2, 35.8, 28.0, 22.7, 20.7, 15.0. IR (film, cm⁻¹): 3492 (NH), 1723 (C═O), 1645 (C═O). HRMS (ESI): Calcd. for C₁₁H₂₀NO₃ ([M+H]⁺) m/z 214.1443, Found 214.1441. GC analysis: CP-Chirasil-Dex CB (Temp program: initial temp=50° C., 10.00° C./min, final temp=200° C., final time=10.00 min) trans-isomer: tₘᵢₙₒᵣ=16.05 min, tₘₐⱼₒᵣ=16.12 min.

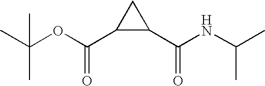

tert-Butyl 2-isopropylaminocarbonyl-cyclopropanecarboxylate (Entry 10, Table 1) was synthesized from N-isopropylacylamide with t-BDA. trans-isomer: ¹H NMR (300 MHz, CDCl₃): δ5.81 (br, 1H), 4.03-4.10 (m, 1H), 2.03-2.09 (m, 1H), 1.78-1.84 (m, 1H), 1.45 (s, 9H), 1.33-1.39 (m, 1H), 1.20-1.25 (m, 1H), 1.18 (d, J=4.2 Hz, 3H), 1.15 (d, J=4.5 Hz, 3H). ¹³C NMR (75 MHz, CDCl₃): δ172.0, 169.4, 80.9, 41.7, 28.0, 24.0, 22.7, 22.3, 14.5. IR (film, cm⁻¹): 3292 (NH), 1724 (C═O), 1643 (C═O). HRMS (ESI): Calcd. for C₁₂H₂₂NO₃ ([M+H]⁺) m/z 228.1600, Found 228.1590. GC analysis: CP-Chirasil-Dex CB (Temp program: initial temp=50° C., 5.00° C./min, final temp=200° C., final time=10.00 min) trans-isomer: tₘᵢₙₒᵣ=28.15 min, tₘₐⱼₒᵣ=28.26 min.

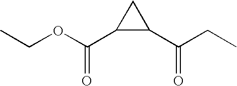

Ethyl 2-propionyl-cyclopropanecarboxylate (Hammerschmidt et al., Annalen der Chemie, 1977, 6, 1026.) (Entry 11, Table 1) was synthesized from ethyl vinyl ketone with EDA. trans-isomer: ¹H NMR (300 MHz, CDCl₃): δ4.15 (q, J=7.2 Hz, 2H), 2.64 (q, J=7.2 Hz, 2H), 2.44-2.48 (m, 1H), 2.14-2.29 (m, 1H), 1.39-1.43 (m, 2H), 1.27 (t, J=7.2 Hz, 3H), 1.08 (t, J=7.2 Hz, 3H). ¹³C NMR (75 MHz, CDCl₃): δ208.1, 172.2, 61.0, 37.1, 28.7, 23.9, 17.0, 14.1, 7.6. IR (film, cm⁻¹): 1729 (C═O), 1707 (C═O). HRMS (ESI): Calcd. for C₉H₁₅O₃ ([M+H]⁺) m/z 171.1021, Found 171.1016. GC analysis: CP-Chirasil-Dex CB (Temp program: initial temp=50° C., Rate₁: 3.00° C./min, max temp=100° C.; Rate₂: 2.00° C./min, max temp=130° C.; Rate₃: 10.00° C./min, max temp=200° C.; final time=5.00 min) trans-isomer: tₘₐⱼₒᵣ=26.42 min, tₘᵢₙₒᵣ=26.84 min.

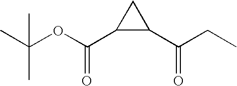

tert-Butyl 2-propionyl-cyclopropanecarboxylate (Entry 12, Table 1) was synthesized from ethyl vinyl ketone with t-BDA. trans-isomer: ¹H NMR (300 MHz, CDCl₃): δ2.64 (q, J=7.2 Hz, 2H), 2.35-2.41 (m, 1H), 2.06-2.12 (m, 1H), 1.45 (s, 9H), 1.32-1.37 (m, 2H), 1.09 (t, J=7.2 Hz, 3H). ¹³C NMR (75 MHz, CDCl₃): δ208.3, 171.2, 81.1, 37.0, 28.5, 28.0, 25.0, 16.9, 7.6. IR (film, cm⁻¹): 1707 (C═O). HRMS (ESI): Calcd. for C₁₁H₂₂O₃N ([M+NH₄]⁺) m/z 216.1600, Found 216.1592. GC analysis: CP-Chirasil-Dex CB (Temp program: initial temp=50° C., Rate₁: 3.00° C./min, max temp=100° C.; Rate₂: 2.00° C./min, max temp=130° C.; Rate₃: 10.00° C./min, max temp=200° C.; final time=5.00 min) trans-isomer: tₘᵢₙₒᵣ=30.11 min, tₘₐⱼₒᵣ=30.40 min.

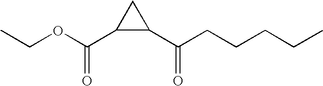

Ethyl 2-hexanoyl-cyclopropanecarboxylate (Ornstein et al., J. Med. Chem. 1998, 41, 346.) (Entry 13, Table 1) was synthesized from 1-octen-3-one with EDA. trans-isomer: ¹H NMR (300 MHz, CDCl₃): δ4.13 (q, J=7.2 Hz, 2H), 2.59 (t, J=7.2 Hz, 2H), 2.41-2.47 (m, 1H), 2.12-2.18 (m, 1H), 1.56-1.66 (m, 2H), 1.39-1.43 (m, 2H), 1.26-1.33 (m, 4H), 1.27 (t, J=7.2 Hz, 3H), 0.89 (t, J=6.9 Hz, 3H). ¹³C NMR (75 MHz, CDCl₃): δ207.7, 172.1, 60.9, 43.9, 31.3, 28.8, 23.9, 23.4, 22.3, 17.0, 14.1, 13.8. IR (film, cm⁻¹): 1731 (C═O), 1706 (C═O). HRMS (ESI): Calcd. for C₁₂H₂₀O₃Na ([M+Na]⁺) m/z 235.1310, Found 235.1305. GC analysis: CP-Chirasil-Dex CB (Temp program: initial temp=50° C., Rate₁: 5.00° C./min, max temp=100° C.; Rate₂: 0.90° C./min, max temp=140° C.; Rate₃: 10.00° C./min, max temp=200° C.; final time=5.00 min) trans-isomer: tₘₐⱼₒᵣ=46.87 min, tₘᵢₙₒᵣ=47.39 min.

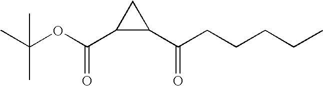

tert-Butyl 2-hexanoyl-cyclopropanecarboxylate (Entry 14, Table 1) was synthesized from 1-octen-3-one with t-BDA. trans-isomer: ¹H NMR (300 MHz, CDCl₃): δ2.59 (t, J=7.5 Hz, 2H), 2.34-2.40 (m, 1H), 2.04-2.10 (m, 1H), 1.56-1.66 (m, 2H), 1.45 (s, 9H), 1.25-1.35 (m, 6H), 0.90 (t, J=6.9 Hz, 3H). ¹³C NMR (75 MHz, CDCl₃): δ208.0, 171.2, 81.0, 43.8, 31.3, 28.6, 28.0, 25.0, 23.4, 22.4, 16.8, 13.8. IR (film, cm⁻¹): 1707 (C═O). HRMS (ESI): Calcd. for C₁₄H₂₄O₃Na ([M+H]⁺) m/z 263.1623, Found 263.1616. GC analysis: CP-Chirasil-Dex CB (Temp program: initial temp=50° C., Rate₁: 5.00° C./min, max temp=100° C.; Rate₂: 2.00° C./min, max temp=150° C.; Rate₃: 10.00° C./min, max temp=200° C.; final time=5.00 min) trans-isomer: tₘᵢₙₒᵣ=37.71 min, tₘᵢₙₒᵣ=37.91 min.

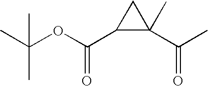

tert-Butyl 2-acetyl-2-methyl-cyclopropanecarboxylate (Entry 15, Table 1) was synthesized from 3-methyl-3-buten-2-one with t-BDA. trans-isomer: ¹H NMR (300 MHz, CDCl₃): δ2.20-2.25 (m, 1H), 2.21 (s, 3H), 1.48 (s, 3H), 1.42-1.50 (m, 1H), 1.46 (s, 9H), 1.24-1.27 (m, 1H). ¹³C NMR (75 MHz, CDCl₃): δ207.5, 169.5, 81.1, 33.6, 30.0, 28.1, 26.8, 21.7, 13.6. IR (film, cm⁻¹): 1728 (C═O). HRMS (ESI): Calcd. for C₁₁H₂₂O₃N ([M+NH₄]⁺) m/z 216.1600, Found 216.1588. GC analysis: CP-Chirasil-Dex CB (Temp program: initial temp=50° C., 4.00° C./min, final temp=200° C., final time=10.00 min) trans-isomer: tₘᵢₙₒᵣ=22.80 min, tₘₐⱼₒᵣ=22.91 min.

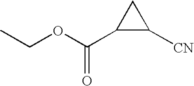

Ethyl 2-cyanocyclopropanecarboxylate (Ashton et al., J. Med. Chem. 1988, 31, 2304.) (Entry 16, Table 1) was synthesized from acrylonitrile with EDA. trans-isomer: ¹H NMR (300 MHz, CDCl₃): δ4.19 (q, J=7.2 Hz, 2H), 2.23-2.30 (m, 1H), 1.91-1.98 (m, 1H), 1.48-1.56 (m, 2H), 1.30 (t, J=7.2 Hz, 3H). ¹³C NMR (75 MHz, CDCl₃): 170.1, 119.2, 61.7, 21.0, 14.4, 14.1, 5.6. IR (film, cm⁻¹): 2245 (CN), 1730 (C═O). HRMS (ESI): Calcd. for C₇H₁₃N₂O₂ ([M+NH₄]⁺) m/z 157.0977, Found 157.0972. cis-isomer: ¹H NMR (300 MHz, CDCl₃): δ4.26 (q, J=7.2 Hz, 2H), 2.09-2.16 (m, 1H), 1.81-1.89 (m, 1H), 1.66-1.72 (m, 1H), 1.38-1.46 (m, 1H), 1.32 (t, J=7.2 Hz, 3H). ¹³C NMR (75 MHz, CDCl₃): δ168.8, 100.1, 61.7, 20.0, 14.1, 13.2, 5.6. IR (film, cm⁻¹): 2245 (CN), 1730 (C═O). HRMS (ESI): Calcd. for C₇H₁₃N₂O₂ ([M+NH₄]⁺) m/z 157.0977, Found 157.0972. GC analysis: G-TA (Temp program: initial temp=50° C., 10.00° C./min, final temp=180° C., final time=10.00 min) trans-isomer: tₘₐⱼₒᵣ=11.69 min, tₘᵢₙₒᵣ=11.83 min; cis-isomer: tₘᵢₙₒᵣ=14.74 min, tₘₐⱼₒᵣ=15.15 min.

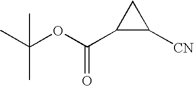

tert-Butyl 2-cyanocyclopropanecarboxylate (Jonczyk A., Makosza M., Synthesis 1976, 6, 387.) (Entry 17, Table 1) was synthesized from acrylonitrile with t-BDA. trans-isomer: ¹H NMR (300 MHz, CDCl₃): δ2.14-2.21 (m, 1H), 1.83-1.90 (m, 1H), 1.38-1.50 (m, 2H), 1.46 (s, 9H). ¹³C NMR (75 MHz, CDCl₃): δ169.1, 119.5, 82.4, 27.9, 22.0, 14.3, 5.3. IR (film, cm⁻¹): 2240 (CN), 1718 (C═O). HRMS (ESI): Calcd. for C₉H₁₇N₂O₂ ([M+NH₄]⁺) m/z 185.1290, Found 185.1286. cis-isomer: ¹H NMR (300 MHz, CDCl₃): δ1.98-2.05 (m, 1H), 1.72-1.81 (m, 1H), 1.58-1.66 (m, 1H), 1.51 (s, 9H), 1.31-1.39 (m, 1H). ¹³C NMR (75 MHz, CDCl₃): δ167.7, 117.8, 82.5, 27.9, 20.9, 12.9, 5.3. IR (film, cm⁻¹): 2241 (CN), 1725 (C═O). HRMS (ESI): Calcd. for C₉H₁₇N₂O₂ ([M+NH₄]⁺) m/z 185.1290, Found 185.1289. GC analysis: CP-Chirasil-Dex CB (Temp program: initial temp=50° C., 10.00° C./min, final temp=200° C., final time=10.00 min) trans-isomer: tₘₐⱼₒᵣ=12.92 min, tₘᵢₙₒᵣ=13.06 min; cis-isomer: tₘᵢₙₒᵣ=13.81 min, tₘₐⱼₒᵣ=13.90 min.

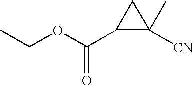

Ethyl 2-cyano-2-methylcyclopropanecarboxylate (Doyle M. P., Davidson J. G., J. Org. Chem. 1980, 45, 1538.) (Entry 18, Table 1) was synthesized from methacrylonitrile with EDA. trans-isomer: ¹H NMR (300 MHz, CDCl₃): δ4.20 (q, J=7.2 Hz, 2H), 2.29-2.34 (m, 1H), 1.58-1.63 (m, 1H), 1.50 (s, 3H), 1.40-1.42 (m, 1H), 1.30 (t, J=7.2 Hz, 3H). ¹³C NMR (75 MHz, CDCl₃): δ168.6, 122.4, 61.5, 26.0, 19.7, 14.7, 14.1, 13.8. IR (film, cm⁻¹): 2243 (CN), 1732 (C═O). cis-isomer: ¹H NMR (300 MHz, CDCl₃): δ4.24 (q, J=7.2 Hz, 2H), 1.81-1.92 (m, 2H), 1.50 (s, 3H), 1.31 (t, J=7.2 Hz, 3H), 1.20-1.25 (m, 1H). ¹³C NMR (75 MHz, CDCl₃): δ168.8, 120.0, 61.7, 27.9, 21.8, 20.9, 14.2, 12.9. IR (film, cm⁻¹): 2243 (CN), 1731 (C═O). HRMS (ESI): Calcd. for C₈H₁₅N₂O₂ ([M+NH₄]⁺) m/z 171.1134, Found 171.1128. GC analysis: CP-Chirasil-Dex CB (Temp program: initial temp=50° C., 10.00° C./min, final temp=200° C., final time=10.00 min) trans-isomer: tₘᵢₙₒᵣ=11.76 min, tₘₐⱼₒᵣ=11.87 min; cis-isomer: tₘᵢₙₒᵣ=12.32 min, tₘₐⱼₒᵣ=12.59 min.

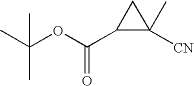

tert-Butyl 2-cyano-2-methylcyclopropanecarboxylate (Jonczyk A., Makosza M., Synthesis 1976, 6, 387.) (Entry 19, Table 1) was synthesized from methacrylonitrile with t-BDA. trans-isomer: ¹H NMR (300 MHz, CDCl₃): δ2.21-2.26 (m, 1H), 1.52-1.58 (m, 1H), 1.50 (s, 3H), 1.47 (s, 9H), 1.33-1.37 (m, 1H). ¹³C NMR (75 MHz, CDCl₃): δ 167.6, 122.7, 82.2, 28.0, 27.2, 19.3, 14.6, 12.2. IR (film, cm⁻¹): 2239 (CN), 1726 (C═O). HRMS (ESI): Calcd. for C₁₀H₁₉N₂O₂ ([M+NH₄]⁺) m/z 199.1447, Found 199.1438. cis-isomer: ¹H NMR (300 MHz, CDCl₃): δ1.73-1.81 (m, 2H), 1.50 (s, 9H), 1.47 (s, 3H), 1.13-1.17 (m, 1H). ¹³C NMR (75 MHz, CDCl₃): δ167.8, 120.2, 82.4, 28.9, 28.0, 21.8, 20.7, 13.4. IR (film, cm⁻¹): 2243 (CN), 1726 (C═O). HRMS (ESI): Calcd. for C₁₀H₁₆NO₂ ([M+H]⁺) m/z 182.1181, Found 182.1172. GC analysis: CP-Chirasil-Dex CB (Temp program: initial temp=50° C., 10.00° C./min, final temp=200° C., final time=10.00 min) trans-isomer: tₘᵢₙₒᵣ=12.63 min, tₘₐⱼₒᵣ=12.70 min; cis-isomer: tₘᵢₙₒᵣ=12.83 min, tₘₐⱼₒᵣ=12.92 min.

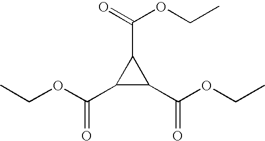

Triethyl trans-1,2,3-cyclopanetricarboxylate (Kozhushkov et al., Synthesis 2003, 6, 956.) (Entry 20, Table 1) was synthesized from diethyl maleate with EDA. ¹H NMR (300 MHz, CDCl₃): δ4.18 (q, J=7.2 Hz, 2H), 4.17 (q, J=7.2 Hz, 4H), 2.77 (t, J=5.7 Hz, 1H), 2.54 (d, J=5.1 Hz, 2H), 1.29 (t, J=7.2 Hz, 3H), 1.27 (t, J=7.2 Hz, 6H). ¹³C NMR (75 MHz, CDCl₃): δ170.1, 167.5, 61.6, 61.5, 28.4, 25.6, 14.0. IR (film, cm⁻¹): 1731 (C═O). HRMS (ESI): Calcd. for C₁₂H₂₂O₆N ([M+NH₄]⁺) m/z 276.1447, Found 276.1435.

General Procedures for Cyclopropanation of Methacrylate. Catalyst (2 mol %) was placed in an oven-dried, resealable Schlenk tube. The tube was capped with a Teflon screwcap, evacuated, and backfilled with nitrogen. The screwcap was replaced with a rubber septum, and 1.0 equivalent of substrate (0.25 mmol) in 0.5 mL chlorobenzene was added via syringe, followed by 1.2 equivalents of diazosulfone compound, followed by the remaining chlorobenzene (0.5 mL). The tube was purged with nitrogen for 1 min and its contents were stirred at room temperature. After the reaction finished, the resulting mixture was concentrated and the residue was purified by flash silica gel chromatography to give the product.

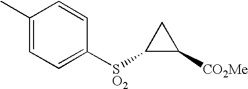

Methyl 2-tosylcyclopropanecarboxylate: Trans-isomer: [α]²⁰_{D}=−46.1 (c=0.40, CHCl₃). ¹H NMR (400 MHz, CDCl₃): δ 7.74 (d, J=8.0 Hz, 2H), 7.34 (d, J=8.0 Hz, 2H), 3.65 (s, 3H), 2.92-2.96 (m, 1H), 2.45-2.50 (m, 1H), 2.43 (s, 3H), 1.67-1.72 (m, 1H), 1.49-1.54 (m, 1H). ¹³C NMR (100 MHz, CDCl₃): δ 170.9, 145.2, 137.0, 130.3, 128.0, 52.7, 40.7, 21.9, 20.1, 13.5. IR (neat, cm⁻¹): 2924, 1732, 1148, 716. HRMS (ESI) ([M+H]⁺) Calcd. for C₁₂H₁₅O₄S: 255.0691, Found 255.0668. HPLC analysis: ee=90%. Chiralcel OD-H (98% hexanes: 2% isopropanol, 1.0 mL/min) trans-isomer: tₘᵢₙₒᵣ=29.4 min, tₘₐⱼₒᵣ=35.3 min.

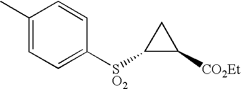

Ethyl 2-tosylcyclopropanecarboxylate: Trans-isomer: [α]²⁰_{D}=−38.2 (c=0.49, CHCl₃). ¹H NMR (400 MHz, CDCl₃): δ 7.75 (d, J=8.0 Hz, 2H), 7.34 (d, J=8.0 Hz, 2H), 4.10 (q, J=7.2 Hz, 2H), 2.91-2.96 (m, 1H), 2.45-2.50 (m, 1H), 2.44 (s, 3H), 1.65-1.70 (m, 1H), 1.48-1.53 (m, 1H), 1.22 (t, J=7.2 Hz, 3H). ¹³C NMR (100 MHz, CDCl₃): δ 170.5, 145.1, 137.0, 130.2, 128.0, 61.8, 40.6, 21.9, 20.3, 14.3, 13.6. IR (neat, cm⁻¹): 2919, 1729, 1149, 716. HRMS (ESI) ([M+H]⁺) Calcd. for C₁₃H₁₇O₄S: 269.0848, Found 269.0849. HPLC analysis: ee=90%. Chiralcel OD-H (99.3% hexanes: 0.7% isopropanol, 2.0 mL/min) trans-isomer: tₘᵢₙₒᵣ=63.4 min, tₘₐⱼₒᵣ=79.5 min.

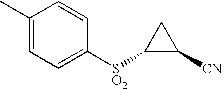

2-Tosylcyclopropanecarbonitrile: Trans-isomer: [α]²⁰_{D}=−28.4 (c=0.29, CHCl₃). ¹H NMR (400 MHz, CDCl₃): δ 7.74 (d, J=8.4 Hz, 2H), 7.38 (d, J=8.4 Hz, 2H), 3.00-3.05 (m, 1H), 2.46 (s, 3H), 2.20-2.24 (m, 1H), 1.80-1.86 (m, 1H), 1.59-1.64 (m, 1H). ¹³C NMR (100 MHz, CDCl₃): δ 146.0, 135.9, 130.6, 128.1, 117.7, 39.3, 21.9, 12.8, 4.8. IR (neat, cm⁻¹): 2248, 1150, 659. HRMS (ESI) ([M+H]⁺) Calcd. for C₁₁H₁₂NO₂S: 222.0589, Found 222.0572. HPLC analysis: ee=61%. Whelk-) 1 (95% hexanes: 5% isopropanol, 1.0 mL/min) trans-isomer: tₘᵢₙₒᵣ=70.5 min, tₘₐⱼₒᵣ=83.6 min.

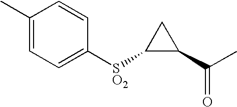

1-(2-Tosylcyclopropyl)ethanone: Trans-isomer: [α]²⁰_{D}=−91.5 (c=0.81, CHCl₃), ee=89%. ¹H NMR (400 MHz, CDCl₃): δ 7.73 (d, J=8.0 Hz, 2H), 7.33 (d, J=8.0 Hz, 2H), 2.90-2.94 (m, 1H), 2.74-2.78 (m, 1H), 2.43 (s, 3H), 2.28 (s, 3H), 1.61-1.66 (m, 1H), 1.44-1.49 (m, 1H). ¹³C NMR (100 MHz, CDCl₃): δ 203.8, 145.1, 137.0, 130.3, 127.9, 42.3, 31.3, 26.5, 21.8, 15.3. IR (neat, cm⁻¹): 1733, 1705, 1144, 732. HRMS (ESI) ([M+H]⁺) Calcd. for C₁₂H₁₅O₃S: 239.0742, Found 239.0738. HPLC analysis: Chiralcel OD-H (98% hexanes: 2% isopropanol, 1.0 mL/min) trans-isomer: tₘᵢₙₒᵣ=35.8 min, tₘₐⱼₒᵣ=39.5 min.

General Procedures for Cyclopropanation of Electron Deficient Olefin: Catalyst (5 mol %) was placed in an oven-dried, resealable Schlenk tube. The tube was capped with a Teflon screwcap, evacuated, and backfilled with nitrogen. The screwcap was replaced with a rubber septum, and 1.25 mmol olefin (dissolve in 1.0 mL PhCl) was added via syringe, followed by 0.25 mmol diazo compound. The tube was purged with nitrogen for 1 min and its contents were stirred at room temperature. After the reaction finished, the resulting mixture was concentrated and the residue was purified by flash silica gel chromatography to give the product. The Cis, Trans-isomers can be separated by column.

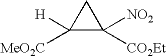

1-Ethyl 2-methyl 1-nitrocyclopropane-1,2-dicarboxylate: Trans-: [α]²⁰_{D}=7.0 (c=0.115, CHCl₃). ¹H NMR (250 MHz, CDCl₃): δ 4.27 (q, J=7.3 Hz, 2H), 3.69 (s, 3H), 3.11-3.03 (m, 1H), 2.17-2.11 (m, 1H), 1.28 (t, J=7.3 Hz, 3H). ¹³C NMR (62.5 MHz, CDCl₃): δ 167.7, 160.7, 63.3, 53.0, 29.6, 21.0, 13.8. HRMS (ESI) ([M+H]⁺) Calcd. for C8H12NO6: 218.0665, Found 218.0655. HPLC analysis: ee=29%. Whelk-O1 (98.5% hexanes: 1.5% isopropanol, 1.0 mL/min) Trans-isomer: tₘᵢₙₒᵣ=12.7 min, tₘₐⱼₒᵣ=15.5 min.

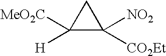

1-Ethyl 2-methyl 1-nitrocyclopropane-1,2-dicarboxylate: Cis-: [α]²⁰_{D}=−40.8 (c=0.155, CHCl₃). ¹H NMR (250 MHz, CDCl₃): δ 4.31-4.21 (m, 2H), 3.69 (s, 3H), 2.83-2.70 (m, 1H), 2.45-2.39 (m, 1H), 1.98-1.90 (m, 1H), 1.29-1.22 (m, 3H). ¹³C NMR (62.5 MHz, CDCl₃): δ 167.3, 164.2, 64.0, 53.2, 29.2, 21.1, 13.9. HRMS (ESI) ([M+H]⁺) Calcd. for C8H12NO6: 218.0665, Found 218.0649. HPLC analysis: ee=80%. Whelk-O1 (98.5% hexanes: 1.5% isopropanol, 1.0 mL/min) Cis-isomer: tₘᵢₙₒᵣ=17.4 min, tₘₐⱼₒᵣ 20.2 min.

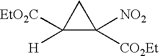

1-Ethyl 2-ethyl 1-nitrocyclopropane-1,2-dicarboxylate: Cis-: [α]²⁰_{D}=−72.9 (c=0.45, CHCl₃). ¹H NMR (250 MHz, CDCl₃): δ 4.29-4.09 (m, 4H), 2.78 (dd, J₁=8.3 Hz, J₂=9.0 Hz, 1H), 2.41 (dd, J₁=6.5 Hz, J₂=7.8 Hz, 1H), 1.92 (dd, J₁=6.3 Hz, J₂=9.3 Hz, 1H), 1.18-1.28 (m, 6H). ¹³C NMR (62.5 MHz, CDCl₃): δ 166.7, 164.3, 71.1, 63.9, 62.4, 29.4, 21.0, 14.0, 13.9. HPLC analysis: ee=88%. OJ-H (99.3% hexanes: 0.7% isopropanol, 0.7 mL/min) Cis-isomer: tₘᵢₙₒᵣ=43.7 min, tₘₐⱼₒᵣ=51.8 min.

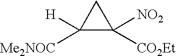

Ethyl 2-(dimethylcarbamoyl)-1-nitrocyclopropanecarboxylate: Cis/trans mixture (Cis/Trans=63/37): [α]²⁰_{D}=−1.65 (c=0.85, CHCl₃). inseparable Cis/Trans mixture: ¹H NMR (250 MHz, CDCl₃): Trans-: δ 4.30-4.20 (m, 2H), 3.24-3.12 (m, 1H), 3.11 (s, 3H), 2.90 (s, 3H), 2.33-2.27 (m, 1H), 2.07-2.00 (m, 1H), 1.28-1.20 (m, 3H). Cis-: δ 4.30-4.20 (m, 2H), 3.11 (s, 3H), 3.00-2.91 (m, 1H), 2.90 (s, 3H), 2.62-2.56 (m, 1H), 1.84-1.78 (m, 1H), 1.28-1.20 (m, 3H). ¹³C NMR (62.5 MHz, CDCl₃): Cis and Trans mixture: δ 164.9, 164.8, 164.5, 161.4, 70.9, 70.6, 63.8, 63.0, 37.4, 37.4, 36.2, 35.9, 29.4, 228.7, 20.6, 20.3, 13.9, 13.8. HPLC analysis: ee (Trans)=22%. Whelk-O1 (95% hexanes: 5.0% isopropanol, 1.0 mL/min) Trans-isomer: tₘₐⱼₒᵣ=44.8, min, tₘᵢₙₒᵣ=58.0 min; Cis-isomer: tₘᵢₙₒᵣ=66.9 min, tₘₐⱼₒᵣ=106.5 min.

The foregoing non-limiting examples are provided to illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

What is claimed is:

1. A process for asymmetric cyclopropanation of an olefin wherein at least one of the olefinic carbon atoms possesses an electron withdrawing group, the process comprising treating the olefin with a diazo reagent in the presence of a chiral porphyrin catalyst.

2. The process of claim 1 wherein the diazo reagent is N₂CHC(O)OR₁₀, 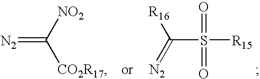
wherein R₁₀ is hydrocarbyl, substituted hydrocarbyl or heterocyclo; R₁₅ and R₁₆ are independently hydrogen, hydrocarbyl or substituted hydrocarbyl; and R₁₇ is hydrogen, hydrocarbyl, substituted hydrocarbyl or heterocyclo.

3. The process of claim 2 wherein the chiral porphyrin catalyst is selected from the group of cobalt porphyrin complexes consisting of

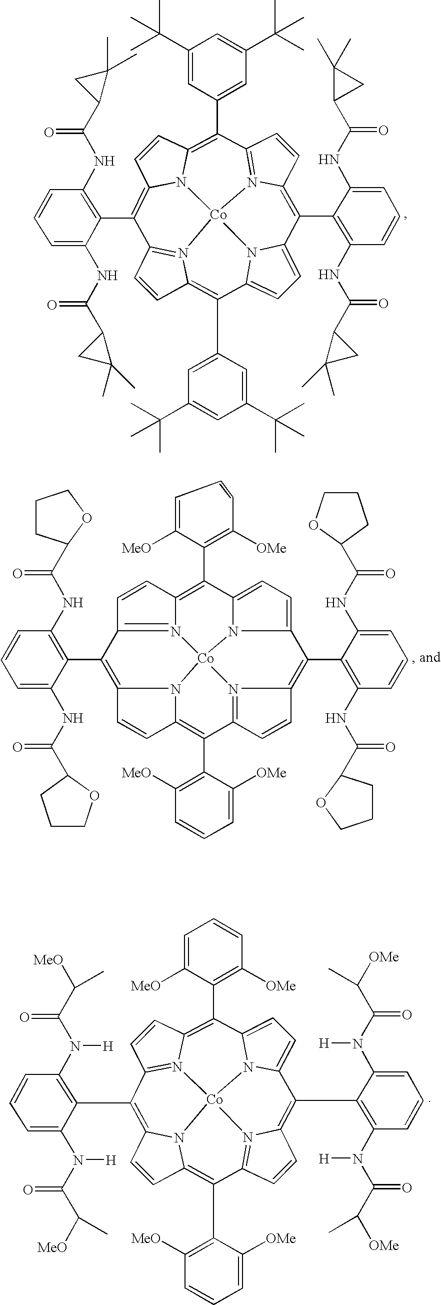

4. The process of claim 2 wherein the olefin corresponds to Formula 1 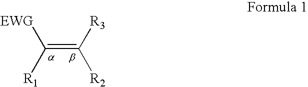
wherein EWG is an electron withdrawing group; R₁ is hydrogen, hydrocarbyl, substituted hydrocarbyl, or heterocyclo; and R₂ and R₃ are independently hydrogen, hydrocarbyl, substituted hydrocarbyl, heterocyclo, or an electron withdrawing group.

5. The process of claim 2 wherein the olefin corresponds to Formula 2 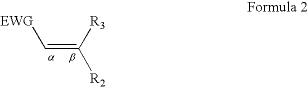
wherein EWG is an electron withdrawing group; and R₂ and R₃ are independently hydrogen, hydrocarbyl, substituted hydrocarbyl, heterocyclo, or an electron withdrawing group.

6. The process of claim 2 wherein the olefin corresponds to Formula 2-trans or Formula 2-cis: 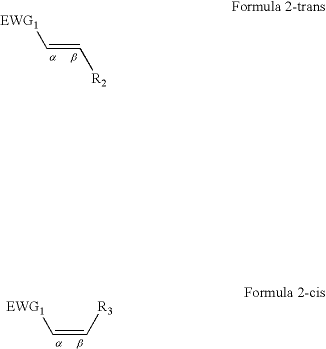
wherein EWG₁ is an electron withdrawing group; R₂ and R₃ are independently hydrogen, hydrocarbyl, substituted hydrocarbyl, heterocyclo, or EWG₂; EWG₂ is an electron withdrawing group; and EWG₁ and EWG₂ are the same or are different.

7. The process of claim 2 wherein the olefin corresponds to Formula 3: 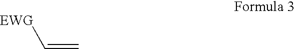
wherein EWG is an electron withdrawing group.

8. The process of claim 2 wherein the electron withdrawing group is an electron withdrawing group selected from the group consisting of hydroxy, alkoxy, mercapto, halo, carbonyl, sulfonyl, nitrile, quaternary amine, nitro, trihalomethyl, imine, amidine, oxime, thioketone, thioester, and thioamide.

9. The process of claim 2 wherein the electron withdrawing group is an electron withdrawing group selected from the group consisting of halo, aldehyde, ketone, ester, carboxylic acid, amide, acid halide, trifluoromethyl, nitrile, sulfonic acid, and nitro.

10. The process of claim 3 wherein the olefin corresponds to Formula 1 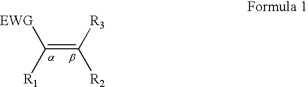
wherein EWG is an electron withdrawing group; R₁ is hydrogen, hydrocarbyl, substituted hydrocarbyl, or heterocyclo; and R₂ and R₃ are independently hydrogen, hydrocarbyl, substituted hydrocarbyl, heterocyclo, or an electron withdrawing group.

11. The process of claim 10 wherein the electron withdrawing group is an electron withdrawing group selected from the group consisting of hydroxy, alkoxy, mercapto, halo, carbonyl, sulfonyl, nitrile, quaternary amine, nitro, trihalomethyl, imine, amidine, oxime, thioketone, thioester, and thioamide.

12. The process of claim 10 wherein the electron withdrawing group is an electron withdrawing group selected from the group consisting of halo, aldehyde, ketone, ester, carboxylic acid, amide, acid halide, trifluoromethyl, nitrile, sulfonic acid, and nitro.

13. The process of claim 1 wherein the diazo reagent corresponds to 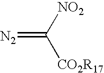
wherein R₁₇ is hydrogen, hydrocarbyl, substituted hydrocarbyl or heterocyclo.

14. The process of claim 13 wherein the olefin corresponds to Formula 1 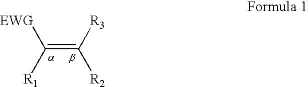
wherein EWG is an electron withdrawing group; R₁ is hydrogen, hydrocarbyl, substituted hydrocarbyl, or heterocyclo; and R₂ and R₃ are independently hydrogen, hydrocarbyl, substituted hydrocarbyl, heterocyclo, or an electron withdrawing group.

15. The process of claim 14 wherein the electron withdrawing group is an electron withdrawing group selected from the group consisting of hydroxy, alkoxy, mercapto, halo, carbonyl, sulfonyl, nitrile, quaternary amine, nitro, trihalomethyl, imine, amidine, oxime, thioketone, thioester, and thioamide.

16. The process of claim 14 wherein the electron withdrawing group is an electron withdrawing group selected from the group consisting of halo, aldehyde, ketone, ester, carboxylic acid, amide, acid halide, trifluoromethyl, nitrile, sulfonic acid, and nitro.

17. The process of claim 1 wherein the diazo reagent corresponds to 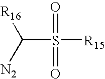
wherein R₁₅ and R₁₆ are independently hydrogen, hydrocarbyl or substituted hydrocarbyl.

18. The process of claim 17 wherein the olefin corresponds to Formula 1 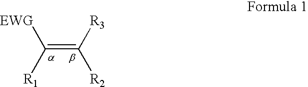
wherein EWG is an electron withdrawing group; R₁ is hydrogen, hydrocarbyl, substituted hydrocarbyl, or heterocyclo; and R₂ and R₃ are independently hydrogen, hydrocarbyl, substituted hydrocarbyl, heterocyclo, or an electron withdrawing group.

19. The process of claim 18 wherein the electron withdrawing group is an electron withdrawing group selected from the group consisting of hydroxy, alkoxy, mercapto, halo, carbonyl, sulfonyl, nitrile, quaternary amine, nitro, trihalomethyl, imine, amidine, oxime, thioketone, thioester, and thioamide.

20. The process of claim 18 wherein the electron withdrawing group is an electron withdrawing group selected from the group consisting of halo, aldehyde, ketone, ester, carboxylic acid, amide, acid halide, trifluoromethyl, nitrile, sulfonic acid, and nitro.
